(19) United States
(12) Patent Application Publication
(10) Pub. No.: US 2016/0045569 A1
(43) Pub. Date: Feb. 18, 2016

(54) ENDOGENOUS VACCINE FOR CANCER AND INFECTIOUS DISEASES

(71) Applicant: NEUMEDICINES, INC., Pasadena, CA (US)

(72) Inventor: Lena A. Basile, Tujunga, CA (US)

(73) Assignee: Neumedicines, Inc., Pasadena, CA (US)

(21) Appl. No.: 14/775,461

(22) PCT Filed: Mar. 13, 2014
(86) PCT No.: PCT/US14/26313

§ 371 Date: Sep. 11, 2015

Related U.S. Application Data
(60) Provisional application No. 61/779,919, filed on Mar. 13, 2013.

(51) Int. Cl. A61K 38/20; A61N 5/10; A61K 39/00; C12N 5/09; A61K 45/06; A61K 35/17

(52) U.S. Cl. CPC A61K 38/208; A61K 45/06; A61K 35/17; A61K 39/0011; C12N 5/0693; A61N 5/10; A61K 2039/5152; C12N 2501/2302; C12N 2501/2304; C12N 2501/2312; C12N 2529/00

(57) ABSTRACT

The present invention is directed to an endogenous vaccine targeted to a cancer or an infectious disease.

### CROSS-REFERENCE TO RELATED APPLICATIONS

This application claims the priority benefits of U.S. provisional patent application 61/779,919, filed Mar. 13, 2013, the entire contents of which are incorporated herein by reference.

### BACKGROUND

The present invention is directed to an endogenous vaccine targeted to a cancer or an infectious disease.

I. Background Regarding Therapeutic Use of Interleukin-12 (IL-12)

IL-12 is a heterodimeric protein composed of p35 and p40 subunits, which can be linked in a recombinant single-chain (p40-p35) IL-12 molecule with retained biological activity. As a multifunctional cytokine, IL-12 bridges innate and adaptive immunity.

It is known that administration of Interleukin-12 (IL-12) facilitates both the recovery of endogenous hematopoiesis and the engraftment of stem cells after ionizing radiation. Burke et al., Exp. Hematol., 35(2):203-13 (February 2007). In addition, it is known that low dose IL-12 can result in multilineage hematopoietic recovery with concomitant antitumor effects in myelosuppressed tumor-bearing mice. Basile et al., J. Transl. Med., 6:26 (May 2008). See also Herodin et al., Exp Hematol., 35:28-33 (2007).

In contrast to a number of pre-clinical studies, early clinical trials using IL-12 as a single agent or as vaccine adjuvant produced only limited efficacy in most instances. Chmielewski et al., Cancer Immunol. Immnother., 61:1269-1277 (2012). In particular, achievement of therapeutic concentrations in the tumor lesion remains elusive because systemic IL-12 administration is limited by dose-dependent toxicity, including toxicity in hematopoietic, intestinal, hepatic, and pulmonary tissues; such toxicity is probably mediated by inducing high IFN-gamma levels. Consequently, clinical trials have explored strategies to deliver IL-12 to the tumor lesion in a locally controlled manner. However, most metastatic cancer lesions are not accessible to direct IL-12 application. This situation favors delivery systems producing the cytokine at the tumor site in situ while avoiding increase in serum concentrations. Chmielewski et al., Cancer Immunol. Immnother., 61:1269-1277 (2012).

IL-12 has a pivotal role in proinflammatory and immunoregulatory functions. It is believed that the antitumor effect of IL-12 is due to improved activation of cytotoxic T cells and NK cells that are the main effector cells of the adaptive and innate immune response in mediating tumor lysis and generating tumor directed antibodies. IL-12, moreover, improves the Th1-type helper T-cell response, induces a panel of cytokines including IFN-γ and TNF-α, and exhibits antiangiogenic activities. These privileges explain the considerable efforts to establish IL-12 in tumor therapy. Clinical trials showed some antitumor effect of IL-12 with Th1-type responses and infiltration of both NK cells and macrophages in the treated tumor lesion. However, some references teach that IL-12 therapy is restricted by severe toxicities preventing systemic administration to achieve therapeutic levels in solid tumor lesions. Chmielewski et al., Cancer Res; 71(17):5697-5706 (Sep. 1, 2011).

For general descriptions relating to IL-12, see U.S. Pat. Nos. 5,573,764, 5,648,072, 5,648,467, 5,744,132, 5,756,085, 5,853,714 and 6,683,046. Interleukin-12 (IL-12) is a heterodimeric cytokine generally described as a proinflammatory cytokine that regulates the activity of cells involved in the immune response (Fitz et al., J. Exp. Med., 170: 827-45 (1989)). Generally IL-12 stimulates the production of interferon-γ (INF-γ) from natural killer (NK) cells and T cells (Lertmemongkolchai et al., J. of Immunology, 166: 1097-105 (2001); Cui et al., Science, 278: 1623-6 (1997); Ohteki et al., J. Exp. Med., 189:1981-6 (1999); Airoldi et al., J. of Immunology, 165: 6880-8 (2000)), favors the differentiation of T helper 1 (TH1) cells (Hsieh et al., Science, 260: 547-9 (1993); Manetti et al., J. Exp. Med., 177: 1199-1204 (1993)), and forms a link between innate resistance and adaptive immunity. IL-12 has also been shown to inhibit cancer growth via its immuno-modulatory and anti-angiogenesis effects (Brunda et al., J. Exp. Med., 178: 1223-1230 (1993)); Noguchi et al., Proc. Natl. Acad. Sci. U.S.A., 93: 11798-11801 (1996); Giordano et al., J. Exp. Med., 194: 1195-1206 (2001); Colombo et al, Cytokine Growth factor, Rev., 13: 155-168 (2002); Yao et al., Blood, 96: 1900-1905 (2000)). IL-12 is produced mainly by dendritic cells (DC) and phagocytes (macrophages and neutrophils) once they are activated by encountering pathogenic bacteria, fungi or intracellular parasites (Reis et al., J. Exp. Med., 186: 1819-1829 (1997); Gazzinelli et al., J. Immunol., 153: 2533-2543 (1994); Dalod et al., J. Exp. Med., 195: 517-528 (2002)). The IL-12 receptor (IL-12 R) is expressed mainly by activated T cells and NK cells (Presky et al., Proc. Natl. Acad. Sci. U.S.A., 93: 14002-14007 (1996); Wu et al., Eur. J. Immunol., 26: 345-50 (1996)).

Generally the production of IL-12 stimulates the production of INF-γ, which, in turn, enhances the production of IL-12, thus forming a positive feedback loop. In in vitro systems, it has been reported that IL-12 can synergize with other cytokines (IL-3 and SCF for example) to stimulate the proliferation and differentiation of early hematopoietic progenitors (Jacobsen et al., J. Exp. Med., 2: 413-8 (1993); Ploemacher et al., Leukemia, 7: 1381-8 (1993); Hirao et al., Stem Cells, 13: 47-53 (1995)).

Other examples of the use of IL-12 are described in US 2013-0259828 for “Uses of IL-12 and the IL-12 receptor positive cell in tissue repair and regeneration;” US 2013-0129674 for “IL-12 formulations for enhancing hematopoiesis;” US 2012-0190909 and US 2011-0206635, both for “Uses of IL-12 in hematopoiesis;” US 2012-0189577 for “Use of IL-12 to increase survival following acute exposure to ionizing radiation;” US 2010-0278777 for “Method for treating deficiency in hematopoiesis;” US 2010-0278778 for “Method for bone marrow preservation or recovery;” and U.S. Pat. No. 7,939,058 for “Uses of IL-12 in hematopoiesis.”

Additionally, HemaMax™, recombinant human interleukin-12 (IL-12), is under development as a front-line radiation medical countermeasure (Rad-MCM) for the treatment of hematopoietic syndrome of ARS (HSARS) due to radiological terrorism or accidental exposure. Basile et al., PLoS ONE, 7 (2): e30434. doi:10.1371/journal.pone.0030434 (2012). HemaMax™ is a potent biologic radiomitigant that increases survival and accelerates recovery following exposure to lethal total body irradiation 24 hrs after exposure. HemaMax™ has recently been shown to be safe in a First-in-Human (FIH) trial and a Phase 1b trial when administered in the effective low-dose therapeutic range required for treatment of HSARS. Toxicology, GMP manufacturing, and Phase 1 safety have been completed for HemaMax™ under the HSARS IND.

To date, there are no endogenous vaccines that can be used to treat infectious diseases and/or cancer. Thus, there is a critical medical need for the discovery and development of such endogenous vaccines.

### SUMMARY OF THE INVENTION

The present invention is directed to an endogenous vaccine that is useful in the treatment of many cancers and infectious diseases. In its broadest terms, the invention comprises two components: (1) a method of generating necrotic and/or apoptotic cells that are diseased, wherein the diseased cells are either cancerous or harboring a pathogen, and (2) administration of Interleukin 12 (IL-12) near the time that necrotic and/or apoptotic cells are generated.

The method of generating the necrotic and/or apoptotic cells generally involves treatment methods that can induce cell damage leading to necrosis and/or apoptosis of diseased cells, such but not limited to as radiation therapy, chemotherapy and surgery. In the present invention, these cell damaging treatment methods, which generate pathogen-associated antigens or tumor associated antigens, yield an endogenous vaccine to the targeted pathogen or cancer when these antigens are generated in conjunction with IL-12 administration.

In general, an IL-12 dose is given at least about 96 hours before or after, or at any time point in-between, the method of generating necrotic and/or apoptotic cells that are diseased. For example, an IL-12 dose can be given at about 90, about 94, about 72, about 68, about 62, about 56, about 48, about 42, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2 hours, about 1 hour, or less than 1 hour before or after the method of generating necrotic and/or apoptotic cells that are diseased.

Examples of methods of generating necrotic and/or apoptotic cells that are diseased include but are not limited to a radiation cell damaging treatment method, a chemotherapy cell damaging treatment method, and a surgical cell damaging treatment method. If IL-12 is being administered in conjunction with a radiation cell damaging treatment method, then IL-12 will be given in repeat doses as radiation is generally fractionated into small, frequent dosing. In one embodiment of the invention, an IL-12 dose is given with each dose of radiation, either before, during, or after administration of a dose of radiation. The IL-12 dose can be given before, during, or after the radiation, with exemplary time points of IL-12 administration being up to about 96 hours before or after initiation of the radiation, or at other time points as described above, e.g., the IL-12 dose can be given about 90, about 94, about 72, about 68, about 62, about 56, about 48, about 42, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2 hours, about 1 hour, or less than 1 hour before or after the initiation of the radiation.

In general, if IL-12 is being administered in conjunction with a chemotherapy cell damaging treatment method, then an IL-12 dose will be given with each cycle of chemotherapy. The IL-12 dose can be given before, during, or after the chemotherapy cycle, with exemplary time points of IL-12 administration being up to about 96 hours before or after initiation of the chemotherapy cycle. In other embodiments of the invention, the IL-12 dose can be given about 90, about 94, about 72, about 68, about 62, about 56, about 48, about 42, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2 hours, about 1 hour, or less than 1 hour before or after the initiation of the chemotherapy cycle.

In general, if IL-12 is being administered in conjunction with a surgical cell damaging treatment method, then the IL-12 dose can be given before, during, or after the surgical treatment, with exemplary time points of IL-12 administration being up to about 96 hours before or after initiation of the surgery. In other embodiments of the invention, the IL-12 dose can be given about 90, about 94, about 72, about 68, about 62, about 56, about 48, about 42, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2 hours, about 1 hour, or less than 1 hour before or after the initiation of surgery.

In another embodiment, encompassed is a dosing schedule of IL-12 for maintenance following administration of the combination therapy of the invention. The IL-12 maintenance dose amount can be any dosage amount as described below, e.g., from about 1 ng/kg up to about 2000 ng/kg, or less than about 2000 ng/kg. In addition, the IL-12 maintenance dose can be administered for any therapeutically effective duration of time. Exemplary IL-12 maintenance dosing periods include, but are not limited to, daily (e.g., one IL-12 dose/day up to yearly (one IL-12 dose/yearly) or any time point in-between, including for example, one IL-12 dose every week, 2 weeks, 3 weeks, 4 weeks, about 5 weeks, about 6 weeks, about 7 weeks, about 8 weeks, about 9 weeks, about 10 weeks, about 11 weeks, about 12 weeks, about 13 weeks, about 14 weeks, or about 15 weeks, 1 month, 2 months, 3 months, 4 months, 5 months, 6 months, 7 months, 8 months, 9 months, 10 months, 11 months, or 12 months. An IL-12 maintenance dose may also be administered for periods of longer than 1 year, e.g., over a several year period.

Each dose of IL-12 administered in the method of the invention is from about 1 ng/kg up to about 2000 ng/kg, or less than about 2000 ng/kg. In other embodiments of the invention, the dose of IL-12 is less than about 1000 ng/kg, less than about 500 ng/kg, about 300 ng/kg, less than about 300 ng/kg, about 200 ng/kg, less than about 200 ng/kg, about 100 ng/kg, less than about 100 ng/kg, about 100 ng/kg or less, about 50 ng/kg or less, or about 10 ng/kg or less. In another embodiment of the invention, IL-12 is given in two or more doses of less than about 50 ng/kg for each dose, or in two or more doses of less than 30 ng/kg for each dose. In yet further embodiments of the invention, an exemplary IL-12 dose range according to the present invention is about 300 ng/kg or less, or about 150 ng/kg or less. In other embodiments of the invention, IL-12 is administered at a dosage of about 400 ng/kg or less, about 375 ng/kg or less, about 350 ng/kg or less, about 325 ng/kg or less, about 300 ng/kg or less, about 275 ng/kg or less, about 250 ng/kg or less, about 225 ng/kg or less, about 200 ng/kg or less, about 175 ng/kg or less, about 150 ng/kg or less, about 125 ng/kg or less, about 100 ng/kg or less, about 75 ng/kg or less, about 50 ng/kg or less, about 25 ng/kg or less, about 20 ng/kg or less, about 15 ng/kg or less, about 10 ng/kg or less, about 5 ng/kg or less, about 4 ng/kg or less, about 3 ng/kg or less, about 2 ng/kg or less, about 1 ng/kg or less, or about 0.5 ng/kg. Exemplary human IL-12 dosages can also include, but are not limited to, about 0.01, about 0.05, about 0.1, about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 μg/dose.

In one embodiment of the invention, the subject to be treated has a cancer which is solid tumor type of cancer, a non-solid tumor type of cancer, a hematopoietic cancer, or a leukemia. Preferred non-solid tumor cancers treatable with the methods of the invention include but are not limited to leukemias. In addition, examples of types of cancer treatable with the methods of the invention include but are not limited to, a solid tumor, carcinomas, sarcomas, lymphomas, cancers that begin in the skin, and cancers that begin in tissues that line or cover internal organs. In another embodiment, examples of such types of cancer include, but are not limited to, brain cancer, including glioblastoma, neuroblastoma, leukemias, lymphomas, thyroid cancer, head and neck cancer, skin cancer, including melanoma, kidney cancer, gastrointestinal cancers, cancer of the digestive system, esophageal cancer, gallbladder cancer, liver cancer, pancreatic cancer, stomach cancer, small intestine cancer, large intestine (colon) cancer, rectal cancer, gynecological cancers, cervical cancer, ovarian cancer, uterine cancer, vaginal cancer, vulvar cancer, prostate cancer, bladder cancer, endometrial cancer, breast cancer, and lung cancer.

In another embodiment of the invention, IL-12 is administered by an injectable delivery route selected from the group consisting of intradermal, intramuscular, intraperitoneal, intravenous, subcutaneous, intratumorally, or epidural routes.

In yet another embodiment of the invention, IL-12 is administered near a site of a tumor or cancer.

The foregoing general description and following description of the drawings and detailed description are exemplary and explanatory and are intended to provide further explanation of the invention as claimed. Other objects, advantages, and novel features will be readily apparent to those skilled in the art from the following brief description of the drawings and detailed description of the invention.

### BRIEF DESCRIPTION OF THE DRAWINGS

FIG. 1 shows a schematic for a model for antigen presentation generated by a treatment that causes necrosis or apoptosis in the presence of IL-12.

FIGS. 2A-C are photomicrographs of mice that received IL-12 plus radiation generates immunity in a lymphoma tumor model. HLA-A-2.1 mice, which are congenic with C57BL/6, had developed large subcutaneous tumors, and then were given curative treatment by surgically removing the tumors. Subsequently, mice were treated with radiation only (A), radiation and IL-12 (B) or radiation, IL-12 and autologous cells from the spleen of a syngenic mouse (C). In (C), the autologous cells were taken from the spleen and cultured ex vivo for about two weeks in the presence of IL-4 to expand the cytotoxic t-lymphocytes (CTL). After 3 months of observation, all mice were re-challenged with the same original dose of EL4 cells. All control mice (A) got large tumors. For animals treated with radiation and IL-12 (B), two out of three of the mice were tumor free with one mouse having a small tumor. All Mice treated with radiation, IL-12 and autologous lymphocytes (C) were tumor free upon rechallenge with tumor cells.

FIG. 3 shows Kaplan-Meier (K-M) plots for murine RmIL-12 for a sucrose-based formulation following subcutaneous (SC) injection with RmIL-12 at 24 hours after exposure to 7.9 Gy. The results show that IL-12 in a sucrose formulation mitigates the effects of lethal irradiation of mice. Control mice received no IL-12 (line labeled “Control”); Treated mice received 18 ng (line labeled “18 ng”) or 162 ng (line labeled “162 ng”) IL-12 in a sucrose-based formulation.

FIG. 4 shows Kaplan-Meier (K-M) plots for murine RmIL-12 for a trehalose-based formulation following subcutaneous (SC) injection with RmIL-12 at 24 hours after exposure to 7.9 Gy. The results show that IL-12 in a trehalose formulation mitigates the effects of lethal irradiation of mice. Control mice received no IL-12 (line labeled “Control”); Treated mice received 2 ng (line labeled “2 ng”) or 18 ng (line labeled “18 ng”) IL-12 in a trehalose-based formulation.

### DETAILED DESCRIPTION OF THE INVENTION

### I. Overview

The present invention has many uses in the treatment of various cancers and infectious diseases. The invention comprises two components that together generate an endogenous vaccine to an existing cancer or pathogen. The two basic components of the endogenous vaccine of the present invention comprise the following: (1) a method of generating necrotic and/or apoptotic diseased cells, whereby the diseased cells are either cancerous or harboring a pathogen, and (2) administration of Interleukin 12 (IL-12) near the time the necrotic and/or apoptotic cells are generated.

Many disease states could be ameliorated, or even cured, via a putative vaccine that would be capable of activating and targeting the immune system of the subject to directly, or indirectly, attack the foreign agents that give rise to the disease. In terms of infectious diseases, such as HIV infection, AIDS or Hepatitis infection, the foreign agents are generally viruses or bacteria that infect the subject and give rise to the particular infectious disease state. Endogenous vaccines could be significant to the eradication of many infectious diseases. In this case, the pathogens would possess pathogenic-associated antigens that could be used to target the disease state. However, other non-infectious disease states that are generated within the subject, such as cancer, can also be treated by an endogenous vaccine. In terms of cancer, the foreign agent is the cancerous cell, which would possess tumor-associated antigens.

To date, there are no endogenous vaccines that can be used to treat infectious diseases and/or cancer. Such vaccines would have many advantages as they would presumably rely on triggering the natural healing mechanisms within the body by natural substances from and/or within the body. Further, an endogenous vaccine should produce fewer side effects than other currently available therapies for the treatment of cancer and infectious diseases. Thus, there is a critical medical need for the discovery and development of such endogenous vaccines.

“Endogenous vaccine,” “vaccine” or “vaccine effect” as referred to herein, is an endogenously generated resistance or immunity to a targeted pathogen or cancer within the body of a subject. Within the meaning of the present invention, an endogenous vaccine is created within the subject by the generation of tumor-associated antigens or pathogen-associated antigens via a disease-related treatment, such as radiation therapy, chemotherapy or surgery, which involves destruction of the cancerous cell or pathogenic cell, generally via necrosis and/or apoptosis. Moreover, in the present invention, IL-12 is acting as an adjuvant to stimulate the immune system to increase the immunological response within the subject to the antigens provided by the treatment. In the present invention a disease-related cancer treatment that generates tumor-associated antigens may be radiation therapy, chemotherapy or surgery, whereas an infectious disease-related treatment that generates pathogen-associated antigens may be radiation therapy or chemotherapy. Thus, the endogenous vaccine of the present invention does not introduce any exogenous cells or exogenous antigens to the subject to generate resistance or immunity to the targeted disease state.

There are two related embodiments of the invention that are used to target a cancer or infectious pathogen within the subject. The methods of the two related embodiments can be used interchangeably, except that in one embodiment cancer is the target of the present invention and in the other embodiment, the target of the present invention is pathogen-containing cells within the body of the subject.

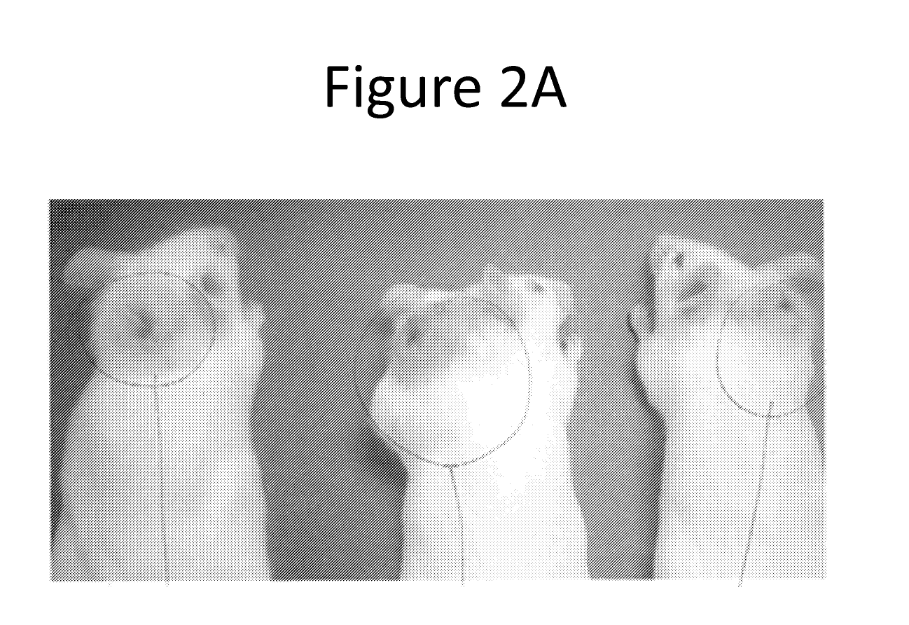

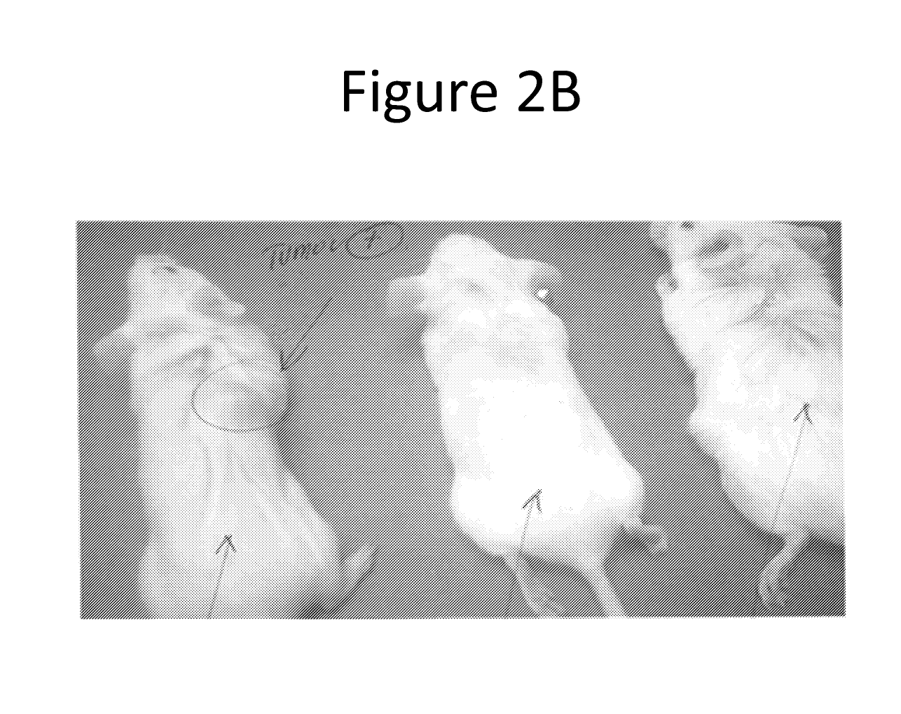

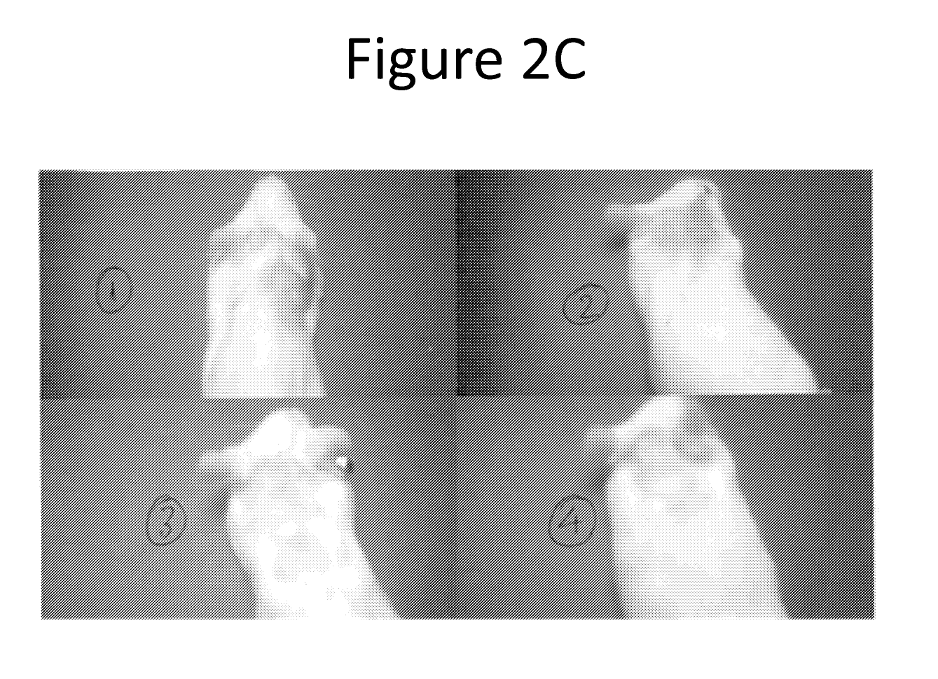

The data described in the examples below support the effectiveness of the endogenous vaccines of the invention. Specifically, Example 1 describes data showing that lymphoma-bearing mice treated with IL-12 and radiation had an average tumor size that was 100× less than that observed with mice treated with radiation alone or IL-12 along: e.g., tumor sizes of about 100 mm³ for lymphoma-bearing mice treated with IL-12 and radiation, as compared to an average tumor size of about 10,000 mm³ for lymphoma-bearing mice treated with radiation alone or IL-12 alone. Moreover, Example 2 describes data demonstrating that administration of IL-12 in conjunction with surgery (e.g., removal of tumors) and radiation provides a protective immune response. Specifically, Example 2 describes an experiment in which large tumors were surgically from mice, followed by radiation in first group and radiation+IL-12 in a second group. Subsequently, the mice were re-challenged (re-inoculated) with lymphoma cells. As shown in FIG. 2A, all of the control mice (3/3) developed large subcutaneous tumors, whereas for the IL-12-treated group shown in FIG. 2(B), only ⅓ of the IL-12-treated mice developed a relatively small tumor, demonstrating at a mimium a 66% increase in a protective immune response, with a likely response even larger as the tumor observed in the IL-12 mouse was very small as compared to the large tumors observed in the non-IL-12 treated group. These data demonstrate that IL-12 can facilitate both hematopoietic recovery and tumor remission in the clinical setting.

### II. Definitions

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

“An associated hematopoietic toxicity” is a toxicity that substantially arises from the administration of the treatment to a mammal that adversely affects the hematopoietic system of the mammal. This adverse effect can be manifested in the mammal broadly whereby many hematopoietic cell types are altered from what is considered to be normal levels, as a result of the treatment, or as a result of the treatment and the disease state combined, or the adverse effect can be manifested in the mammal more specifically whereby only one or a few hematopoietic cell types are altered from what is considered to be normal levels, as a result of the treatment, or as a result of the treatment and the disease state combined.

“Chemotherapy” refers to any therapy that includes natural or synthetic agents now known or to be developed in the medical arts. Examples of chemotherapy include the numerous cancer drugs that are currently available. However, chemotherapy also includes any drug, natural or synthetic, that is intended to treat a disease state. In certain embodiments of the invention, chemotherapy may include the administration of several state of the art drugs intended to treat the disease state. Examples include combined chemotherapy with docetaxel, cisplatin, and 5-fluorouracil for patients with locally advanced squamous cell carcinoma of the head (Tsukuda et al., Int. J. Clin. Oncol., 9(3):161-6 (June 2004)), and fludarabine and bendamustine in refractory and relapsed indolent lymphoma (Konigsmann et al., Leuk. Lymphoma, 45(9):1821-1827 (2004)). Another example is the current treatment for HIV infection, or AIDS, currently referred to as HAART, that involves administering at least three antiviral agents to a patient as a treatment for HIV infection. Still another type of chemotherapy within the scope of the invention are antibiotics and antivirals used to treat pathogenic infections.

“Disease state” refers to a condition present in a mammal whereby the health and well-being of the mammal is compromised. In the present invention, various forms of cancer and various infectious diseases are the targeted disease states of the endogenous vaccine of the invention. In certain embodiments of the invention, treatments intended to target the disease state are administered to the mammal.

“Interleukin-12 (IL-12)” refers to any IL-12 molecule that yields at least one of the properties disclosed herein, including native IL-12 molecules, variant 11-12 molecules and covalently modified IL-12 molecules, now known or to be developed in the future, produced in any manner known in the art now or to be developed in the future. Generally, the amino acid sequences of the IL-12 molecule used in embodiments of the invention are derived from the specific mammal to be treated by the methods of the invention. Thus, for the sake of illustration, for humans, generally human IL-12, or recombinant human IL-12, would be administered to a human in the methods of the invention, and similarly, for felines, for example, the feline IL-12, or recombinant feline IL-12, would be administered to a feline in the methods of the invention. Also included in the invention, however, are certain embodiments where the IL-12 molecule does not derive its amino acid sequence from the mammal that is the subject of the therapeutic methods of the invention. For the sake of illustration, human IL-12 or recombinant human IL-12 may be utilized in a feline mammal. Still other embodiments of the invention include IL-12 molecules where the native amino acid sequence of IL-12 is altered from the native sequence, but the IL-12 molecule functions to yield the properties of IL-12 that are disclosed herein. Alterations from the native, species-specific amino acid sequence of IL-12 include changes in the primary sequence of IL-12 and encompass deletions and additions to the primary amino acid sequence to yield variant IL-12 molecules. An example of a highly derivatized IL-12 molecule is the redesigned IL-12 molecule produced by Maxygen, Inc. (Leong et al., Proc. Natl. Acad. Sci. USA., 100(3): 1163-8 (2003)), where the variant IL-12 molecule is produced by a DNA shuffling method. Also included are modified IL-12 molecules are also included in the methods of invention, such as covalent modifications to the IL-12 molecule that increase its shelf life, half-life, potency, solubility, delivery, etc., additions of polyethylene glycol groups, polypropylene glycol, etc., in the manner set forth in U.S. Pat. Nos. 4,640,835; 4,496,689; 4,301,144; 4,670,417; 4,791,192 or 4,179,337. One type of covalent modification of the IL-12 molecule is introduced into the molecule by reacting targeted amino acid residues of the IL-12 polypeptide with an organic derivatizing agent that is capable of reacting with selected side chains or the N- or C-terminal residues of the IL-12 polypeptide. Both native sequence IL-12 and amino acid sequence variants of IL-12 may be covalently modified. Also as referred to herein, the IL-12 molecule can be produced by various methods known in the art, including recombinant methods. Since it is often difficult to predict in advance the characteristics of a variant IL-12 polypeptide, it will be appreciated that some screening of the recovered variant will be needed to select the optimal variant. A preferred method of assessing a change in the properties of variant IL-12 molecules is via the lethal irradiation rescue protocol disclosed below. Other potential modifications of protein or polypeptide properties such as redox or thermal stability, hydrophobicity, susceptibility to proteolytic degradation, or the tendency to aggregate with carriers or into multimers are assayed by methods well known in the art.

Exemplary IL-12 formulations are described, for example, in U.S. Pat. No. 7,939,058, US 2011-0206635, and US 2012-0190909, all for “Uses of IL-12 in Hematopoiesis;” US 2010-0278777 for “Method for Treating Deficiency in Hematopoiesis;” US 2010-0278778 for “Method for Bone Marrow Preservation or Recover;” US 2012-0189577 for “Use of IL-12 to Increase Survival Following Acute Exposure to Ionizing Radiation;” US 2013-0129674 and WO 2011/146574, both for “IL-12 Formulations For Enhancing Hematopoiesis;” US 2013-0259828 and WO 2012/050829, both for “Uses Of IL-12 And The IL-12 Receptor Positive Cell In Tissue Repair And Regeneration;” WO 2012/174056 for “Mitigation Of Cutaneous Injury With IL-12;” and WO 2013/016634 for “Use Of IL-12 To Generate Endogenous Erythropoietin,” all of which are specifically incorporated by reference.

“One or more therapeutically effective dose(s) of IL-12” is any dose administered at any time intervals and for any duration that can substantially generate the endogenous vaccine effect in the subject. In the invention, IL-12 can be viewed as an adjuvant, whereas radiation therapy or chemotherapy serve to generate antigen endogenously via necrosis and/or apoptosis of cancerous cells or cells that are harboring a pathogen.

“Near the time of administration of the treatment” refers to the administration of IL-12 at any reasonable time period either before and/or after the administration of the treatment, such as one month, three weeks, two weeks, one week, several days, one day, 20 hours, several hours, one hour or minutes, or at other time points as described herein. Near the time of administration of the treatment may also refer to either the simultaneous or near simultaneous administration of the treatment and IL-12, i.e., within minutes to one day.

“Hematopoietic disorders (cancers)” generally refers to the presence of cancers of the hematopoietic system such, as leukemias, lymphomas etc.

“HIV infection” refers any stage of viral infection or exposure, regardless of the presence of symptoms of HW infection or AIDS. Further, herein HIV infection refers to the harboring of the HIV virus within cells of a mammal.

“Hepatitis infection” refers to any type of infection generated by one or more forms of the hepatitis virus, referred to as viral hepatitis.

“Hematopoietic stem cells” are generally the blood stem cells; there are two types: “long-term repopulating” as defined above, and “short-term repopulating” which can produce “progenitor cells” for a short period (weeks, months or even sometimes years depending on the mammal); these are also referred to herein as hematopoietic repopulating cells.

“Hematopoietic progenitor cells” are generally the first cells to differentiate from (i.e., mature from) blood stem cells; they then differentiate (mature) into the various blood cell types and lineages.

“Radiation or radiation therapy or radiation treatment” refers to any therapy where any form of radiation is used to treat the disease state. The instruments that produce the radiation for the radiation therapy are either those instruments currently available or to be available in the future.

“Solid tumors” generally are manifested in various cancers of body tissues, such as solid tumors manifested in lung, breast, prostate, ovary, etc., and are cancers other than cancers of blood tissue, bone marrow or the lymphatic system.

“A treatment” is intended to target the disease state and combat it, i.e., ameliorate the disease state. The particular treatment thus will depend on the disease state to be targeted and the current or future state of medicinal therapies and therapeutic approaches. A treatment may have associated toxicities.

### III. Embodiments Related to an Endogenous Cancer Vaccine

The present invention includes embodiments directed to an endogenous cancer vaccine, which is generated endogenously within the body of the subject. The generation of the endogenous cancer vaccine of the present invention generally comprises two components, but may include other components. These two components are: (1) one or more cancer treatments which are administered to a subject who has a cancer, and (2) a therapeutically effective dose of IL-12, preferably administered within about 96 hours of the one or more cancer treatments, wherein administration of the one or more cancer treatments and IL-12 endogenously generates immunity-related cells and molecules in the subject. The vaccine can further comprise the addition of alpha interferon.

The cancer can be, for example, a solid tumor, a hematopoietic cancer, or a combination thereof, or as otherwise described herein.

Additionally, for the endogenous cancer vaccine, the administration of IL-12 can reduce the hematological toxicity of the cancer treatment. This is significant, as the cancer treatment can cause necrosis and/or apoptosis of cells within the subject that harbor the cancer.

The components when administered to a subject who has cancer produce an endogenous vaccine can result in: (a) resistance to the cancer; (b) a treatment for the primary cancer; (c) prevention of metastasis; (d) treatment of one or more metastases; (e) prolonging cancer remission in subjects with cancer; (f) a reducing in the incidence of new tumors in the subject at about 3 months post-treatment with IL-12 as compared to a subject receiving the same cancer treatment but which does not receive IL-12; (g) a decrease in the average volume of tumors in the subject at about 3 months post-treatment with IL-12 as compared to that of a subject receiving the same cancer treatment but which does not receive IL-12; or (h) any combination thereof. In some embodiments of the invention, the incidence of new tumors at about 3 months post-treatment, as compared to a subject who has received the same cancer treatment but not IL-12, is decreased by about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, or about 75% or more. In other embodiments of the invention, the decrease in the average volume of tumors in the subject at about 3 months post-treatment with IL-12 as compared to that of a subject receiving the same cancer treatment but which does not receive IL-12 is about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, or about 75% or more.

The subject who is to receive an embodiment of the endogenous vaccine of the present invention is a generally a mammal, preferably a human. Embodiments of the present invention further include repeated administration, i.e., more than one administration of IL-12, at certain time intervals following the initial administration. Additionally, the two components can be administered more than once to achieve the desired effect of cancer resistance or immunity, as well as eradication of the cancerous tissues and cells from the subject. Also each component may be administered more than once with or without the other component to achieve the desired therapeutic effects. Subsequent doses of IL-12 may be the same or different from the initial dose.

IL-12 can be administered to the subject in many ways. These methods of administration include intravenous, subcutaneous, intraperitoneal, intradermal, or the like. Another method of administration of IL-12 is via continuous infusion. The continuous infusion method has the advantage of delivery a low dose of IL-12 over longer time period, which can add to the effectiveness of present invention.

Generally the two components of the invention are administered to the subject at some time interval relative to one another that is somewhat close in time as described herein, such as within about 96 hours of each other, within about 36 hours of each other, within about 24 hours of each other, within about 12 hours of each other, within about 6 hours of each other, within about 3 hours of each other, and within about one hour of each other. Preferably the two components of the invention are administered within about 24 hours of each other or at other time points as described herein.

Another embodiment consists of IL-12 being administered both before and after the cancer treatment at certain time intervals as described herein. For the administration of IL-12 following the cancer treatment, consideration of the lifetime of the chemotherapy agent or active metabolites, or the lifetime of the radiation-induced endogenous intermediates, should be given. For example, when the chemotherapeutic agent is cyclophosphamide, the preferred time point for IL-12 administration following treatment with cyclophosphamide is at least about 24 hours after the administration of chemotherapy (or at time points as described herein after about 24 hours following chemotherapy treatment), such as about 48 hours or more after the administration of chemotherapy. For radiation, since the radiation-induced endogenous intermediates are generally short-lived, the post-radiation dose of IL-12 is preferentially administered shortly after radiation, preferably within about 10 hours or less, such as 6 hours or less, or at other time points as described herein.

Chemotherapeutic agents compatible with the present invention include all currently available chemotherapies as well as chemotherapeutic agents to be developed in the future. Examples of chemotherapeutic agents compatible with the present invention include but are not limited to cyclophosphamide, etoposide, carboplatin, cisplatin, paclitaxel, abraxane, adriamycin, bleomycin, or the like. A list of representative chemotherapeutic agents compatible with the present invention is shown in Table 1.

TABLE 1
Exemplary Chemotherapeutic Agents for Use with the Invention

| DRUG | MECHANISM | ADME | USES | A/E | MISC |
|---|---|---|---|---|---|
| ALKYLATING | | | | | not cell-cycle phase |
| AGENTS | | | | | specific |
| Cyclophosphamide | cross-linking of DNA | oral | broad general | MYELOSUPPRESSION | resistance via. |
| (Cytoxan) | via. covalent bond of | pro-drug, must 1^{st} be | activity | mucosal damage to GI tract | glutathione conjugation, |
| | akyl group to DNA | metabolized by CYP450 into | solid tumors | HEMORRHAGIC | enhanced DNA repair |
| | template (esp. rxn w/ 7- | reactive cytotoxic metabolites | | CYSTITIS (via. acrolein | mechanisms or increased |
| | nitrogen of guanine) | i.e. acrolein | | metabolite) - keep pts well | metabolism to inactive |
| | | | | hydrated, administer Mesna | metabolites |
| | | | | cardiotoxicity - monitor pts | acquired resistance does |
| | | | | w/ ECGs | not imply cross resistance |
| | | | | SIADH - ↓serum Na+ and | |
| | | | | CNS toxicity, can get | |
| | | | | seizures | |
| Ifosfamide | cross-linking of DNA | oral | testis, sarcomas | MYELOSUPPRESSION | |
| | (esp. rxn w/ 7-nitrogen | activated by hydroxylation in | solid tumors | mucosal damage to GI tract | |
| | of guanine) | liver (slower than | | hemorrhagic cystitis (via. | |
| | | cyclophosphamide) to | | chloracetaldehyde | |
| | | dechlorinated metabolite and | | metalbolite) | |
| | | chloracetaldehyde (toxic | | keep pts well hydrated, | |
| | | metabolite) | | administer Mesna | |
| | | | | CNS toxicity w/ intrathecal | |
| | | | | administration | |
| | | | | encephalopathy | |
| PLATINUM BASED | | | | | |
| CMPDS | | | | | |
| Cisplatin | covalently binds DNA | nonenzymatic inactivation | lung, testis, | NEPHROTOXICITY w/ Mg | resistance via. enhanced |
| | bases and disrupts | intracellularly and in the | ovarian, bladder | wasting, hypokalemia - | DNA repair |
| | DNA function via. | bloodstream by sulfhydryl | | monitor electrolytes and | |
| | crosslinking w/ | groups | | maintain high urine flow | |
| | platinations | highly covalently bound to | | N/V - prophylactic anti- | |
| | | proteins | | emetics | |
| | | >90% renal elimination of | | PERIPHERAL | |
| | | bound drug (adjust dose w/ | | NEUROPATHY, “stocking | |
| | | renal insufficiency) | | and glove” parasthesia, | |
| | | | | usually reversible | |
| | | | | OTOTOXICITY, permanent | |
| | | | | mild myelosuppression | |
| | | | | rare hypersensitivity rxn | |
| Carboplatin | covalently binds DNA | | ovarian, lung, | MYELOSUPPRESSION | not cross resistance with |
| | bases and disrupts | | bladder | (esp. WBCs) | cisplatin |
| | DNA function via. | | | less N/V | |
| | crosslinking | | | less nephrotoxicity | |
| ANTIMETABOLITES | | | | | |
| Methotrexate (MTX) | Folic Acid Analog | oral, intrathecal, IV, IM | bladder, | myelosuppression | rescue BM and mucositis |
| | inhibits dihydrofolate | requires active transport into | lymphoma, | mucositis, GI epithelial | w/ Leucovorin |
| | reductase | cell | brain, tumors, | denudation | do not administer to pts |
| | (DHFR) → depletes | metabolized to | osteosarcoma, | nephrotoxicity (prevent w/ | w/ poor renal fcn; monitor |
| | reduced folates | polyglutamates in normal and | chilhood ALL | hydration) | creatinine clearance |
| | inhibits DNA and RNA | malignant tissues | | pneumonitis | resistance via. increased |
| | synthesis | doesn't cross BBB (can | | neurotoxicity (if intrathecal | DHFR prodxn or |
| | mainly active during | administer intrathecally) | | or high doses) | decreased affinity for |
| | S-phase - | binds plasma prots but is | | hepatotoxicity | MTX or decreased |
| | give as 4 or 24 hour | displace by aspirin and other | | teratogenic | cellular uptake |
| | continuous infusion | drugs | | | inhibits cell-mediated |
| | | eliminated intact in urine; | | | immune rxns - used at low |
| | | urine alkalinization promotes | | | doses for rheumatoid |
| | | excretion (limited solubility in | | | arthritis, Crohn's dz |
| | | acidic environment) | | | |
| 5-Fluorouracil (5-FU) | Pyrimidine Analog of | IV (topical for skin Ca) | slow growing | MYELOSUPPRESSION | cytotoxicity increased by |
| | dUMP | prodrug that must be | solid tumors | (if bolus) | Leucovorin in tx of colon |
| | inhibits thymidylate | converted to nucleotide from | breast, all GI | MUCOSITIS (w/ continuous | cancer, also increase |
| | synthetase → depletion | (F-dUMP) by liver | (esp. colon), | infusion) & hand-foot | toxicity |
| | of thymidine | crosses BBB | bladder, breast, | syndrome of erythematous | resistance via. decrease |
| | phase specific to GI and | metabolized in liver | head & neck | desquamation | in drug activation or use |
| | S | excreted in lungs and urine | premalignant | cardiac toxicity | of alternate pathways |
| | inhibits DNA if given | | lesions (topical) | hepatic toxicity | |
| | as continuous infusion | | | diarrhea, stomatitis | |
| | inhibits RNA if given | | | | |
| | as a bolus | | | | |
| Cytarabine | Pyrimidine Analog of | IV or intrathecal | acute | MYELOSUPPRESSION | resistance via. decrease |
| | dCTP/dCDP | must be phosphorylated to tri- | leukemias | alopecia | in drug activation, |
| | inhibits nucleotide | P active form in the tumor | | N/V | increase in drug |
| | diphosphate reductase, | rapidly inactivated by hepatic | | neurotoxic (high dose) | inactivation, use of |
| | DNA polymerase | cytidine deaminase in the | | | alternate pathways |
| | S phase specific | plasma therefore administered | | | |
| | | as a continuous IV infusion | | | |
| Gemcitibine | Pyrimidine Analog | must be phoshorylated to | bladder, | mild myelosuppression | |
| | inhibits ribonucleotide | mono-P | pancreas, lung | mild flu-like sxs | |
| | reductase → DNA | | | rashes | |
| | strand termination | | | | |
| | sensitizes cells to | | | | |
| | radiation therapy | | | | |
| NATURAL | | | | | |
| PRODUCTS | | | | | |
| Vinblastine | binds tubulin and | IV | Hodgkin's and | MYELOSUPPRESSION | no cross resistance b/w |
| | prevents microtublule | metabolized in liver | non-Hodgkin's | | vinca alkaloids |
| | assembly → arrest in | excreted in bile | lymphoma, | | resistance via. MDR drug |
| | metaphase | | breast | | efflux pump |
| | | | w/ bleomycin | | |
| | | | for testicular | | |
| | | | tumors | | |
| Vincristine | binds tubulin and | IV | ALL, Hodgkin's | PERIPHERAL | minimal |
| | prevents microtublule | metabolized in liver | and non- | NEUROPATHY | myelosuppression |
| | assembly → arrest in | excreted in bile | Hodgkin's | SIADH | resistance via. MDR drug |
| | metaphase | longest half life of vincas → | lymphoma | | efflux pump |
| | | lowest max tolerated dose | | | |
| | | IV, never intrathecal b/c fatal | | | |
| Vinorelbine | binds tubulin and | metabolized in liver | | MYELOSUPPRESSION | newer and less toxic |
| | prevents microtublule | excreted in bile | | mild neuropathy | |
| | assembly → arrest in | | | | |
| | metaphase | | | | |
| Paclitaxol (Taxol) | binds tubulin and | limited water solubility | ovary, breast, | MYELOSUPPRESSION, | resistance via. mutations |
| | prevents microtublule | therefore formulated with | bladder, | esp. neutropenia and | in alpha or beta subunits |
| | depolymerization → | ethanol | prostate | thrombocytopenia | of tubulin |
| | no mitosis | hepatic metabolism and | | alopecia | anaphylactic rxn avoided |
| | | biliary excretion | | N/V | by prophylaxis w/ |
| | | | | sensory neuropathy | antihistamine (i.e. |
| | | | | PERIPHERAL | diphenhydramine/ |
| | | | | NEUROPATHY | Benadryl + steroid/ |
| | | | | ANAPHYLAXIS | hydrocortisone |
| | | | | myalgias | |
| | | | | cardiac toxicity | |
| Docitaxel (Taxotere) | binds tubulin and | hepatic metabolism and | breast, prostate | myelosuppression, esp. | resistance via. mutations |
| | prevents microtublule | biliary excretion | | neutropenia and | in alpha or beta subunits |
| | depolymerization → | | | thrombocytopenia | of tubulin |
| | no mitosis | | | alopcecia | anaphylactic rxn avoided |
| | | | | N/V | by prophylaxis w/ |
| | | | | sensory neuropathy | antihistamine (i.e. |
| | | | | less neurotoxicity | diphenhydramine/ |
| | | | | capillary leak syndrome → | Benadryl + steroid/ |
| | | | | pleural effusion and periph | hydrocortisone |
| | | | | edema | |
| Etoposide | inhibits topoisomerase | IV or parenteral | lung, testis, | MYELOSUPPRESSION | resistance via. MDR drug |
| | II → DNA strand | | lymphomas | N/V | efflux pump |
| | breakage | | | alopecia | |
| | arrests cells in S and | | | mucositis | |
| | early G2 | | | 2^{nd}ary leukemias | |
| Irinotecan | targets topoisomerase | prodrug must be converted | colon | DELAYED ONSET | |
| | I and causes double | to active metabolite | | DIARRHEA | |
| | stranded DNA breaks | | | myelosuppression | |
| | | | | N/V | |
| | | | | alopecia, low fevers | |
| Doxorubicin | Anthracycline antibiotic | IV | broad general | CARDIAC TOXICITY - | reduce cardiac toxicity w/ |
| (Adriamycin) | inhibits RNA synthesis | elimination via. liver, bile, | activity (no | chronic congestive | dexrazoxane or with an |
| | binds dsDNA and | aglycone formation and others | colon, lung) | cardiomyopathy via. | iron chelator |
| | intercalates b/w GC | | | generation of free radicals | can sensitize nml tissue |
| | pairs | | | in mycocardial cells | to radiation |
| | inhibits function of | | | myelosuppression | increased risk of cardiac |
| | topoisomerase II → | | | MUCOSITIS (esp. if | fx for pts w/ HTN, |
| | DNA damage | | | continuous infusion) | preexisting heart dz |
| | free radical formation | | | alopecia | resistance via. MDR drug |
| | via. lipid peroxidation | | | skin necrosis if extravasation | efflux pump |
| | interacts w/ cell | | | urine discoloration | |
| | membrane | | | | |
| Bleomycin | Antibiotic | IV, IM, SQ | squamous cell | PULMONARY FIBROSIS | v. minimal |
| | nicks DNA and inhibits | renal elimination | carcinomas of | rare hypersensitivity rxn | myelosuppression |
| | DNA ligase →DNA | | the head, neck, | | |
| | fragmentation | | skin, esophagus | | |
| | free radical formation | | and GU tract; | | |
| | most active during G2 | | Hodgkin's and | | |
| | and M phases | | non-Hodgkin's | | |
| | | | lymphomas | | |
| | | | w/ vinblastine | | |
| | | | or etoposide | | |
| | | | for testicular | | |
| | | | tumors | | |

One of the therapeutic effects of the present invention is to generate immunity to the targeted cancer. Herein the targeted cancer means the cancer for which the subject is being treated. The endogenous immunity that is generated can have multiple effects: Some of these effects are: (1) to reduce the primary cancerous lesion, and (2) to reduce the reoccurrence of the targeted cancer (reduction of micrometastases, i.e., reduction of minimal residual disease (MRD)), as well as prevention of metastasis. Further, the endogenous vaccine of the present invention may also render the subject resistant to other forms of cancer other than the targeted cancer. This effect would depend on whether the tumor associated antigens (TAA) that are generated from cancer cells following the administration of the cancer treatment are tumor specific antigens or generalized antigens that are found in more than one cancer. If these antigens are generalized antigens, there is an expectation that the endogenous vaccine would create resistance to other forms of cancer, other than the targeted cancer. Table 2 shows some tumor-associated antigens, which may be specific or generalized antigens.

TABLE 2
Examples of human tumor-associated antigens recognized by T cells*

| Category | Gene† | Tumor Expression |
|---|---|---|
| Cancer test | BAGE | Melanoma, myeloma, lung, bladder, |
| | | and breast carcinoma |
| | GAGE-1 | Melanoma, myeloma, lung, bladder, |
| | | prostate, and breast carcinoma, |
| | | esophageal and head/neck SCC, |
| | | sarcoma |
| | MAGE-A1 | Melanoma, myeloma, lung, bladder, |
| | | prostate, colorectal, and breast |
| | | carcinoma, esophageal and head/neck |
| | | SCC, sarcoma |
| | NY-ESO-1 | Melanoma, myeloma, lung, bladder, |
| | | prostate, and breast carcinoma, |
| | | esophageal and head/neck SCC, |
| | | sarcoma |
| Differentiation | GP100 | Melanoma |
| | Mean-A/ | Melanoma |
| | MART-1 | |
| | Prostate-specific | Prostate Carcinoma |
| | Antigen | |
| | Mammoglobin- | Breast carcinoma |
| | A | |
| Overexpressed | Alpha- | Hepatocelluar carcinoma and |
| | fetoprotein | yolk-sac tumors |
| | HER-2/neu | Melanoma, ovarian, gastric, |
| | | pancreatic, and breast carcinoma |
| | P53 | Esophageal, gastric, colon, |
| | | pancreatic, and other carcinomas |
| Mutated (shared) | K-ras | Pancreatic and colorectal |
| † | | adenocarcinomas |
| | TRP-2/INT2 | Melanoma, high grade gliomas |

Abbreviation:
SCC = squamous cell carcinoma.
*This table lists only some examples of the more common tumor antigens identified. For references about individual antigens listed and for a comprehensive review see Novellino et al., Can. Immunol. Immunother., 54: 187-207 (2005).
†Mutated antigens are tumor-specific.
However, few mutations common to more than one patient and sometimes more than one tumor type have been identified. These mutations are usually crucial in the process of neoplastic transformation. Table information extracted from 656 I. J. Radiation Oncology • Biology • Physics Volume 63, Number 3, 2005.

In terms of generating immunity or resistance to a targeted cancer, the antigen specificity of T and B cells of the immune system (i.e., their ability to recognize with extreme specificity the subtle differences that occur in normal cells upon infection or transformation) is an effect in the present invention. Truly tumor specific antigens (TSA) are generally rare but do exist. They can arise from point mutations or other genetic alterations specific to a given tumor or group of tumors, such as fusion proteins generated by translocations, or sometimes from alterations in posttranslational modification. Most of the tumor antigens that are targets for the immune system are more properly defined as tumor-associated antigens (TAA) (see Table 2). This definition includes antigens that are not mutated but are differentially expressed by neoplastic and normal cells, either in time, quantity, location, or cellular context, resulting in a preferential or exclusive recognition of the tumor by the immune system. For example, carcinoembryonic antigens are normally expressed only during embryonic development, p53 and HER-2/neu are overexpressed in some cancer cells. Another example of tumor-associated antigens are represented by a growing family of cancer/testis antigens that are expressed only in male germ cells, and sometimes in placenta and fetal ovary. Tumor-associated antigens with a tissue-restricted expression can be legitimate targets for immunotherapy, especially when the tumor arises from nonessential tissues, such as differentiation antigens expressed by melanoma, and prostate cancer. A special class of TAA is derived from oncogenic viruses associated with some types of cancer, such as human papilloma virus E6 and E7 proteins in cervical cancer, and Epstein-Barr virus—derived antigens in lymphomas.

TAA-specific T cells are frequently detected in the peripheral blood and within the tumor of cancer patients. Tumor-infiltrating lymphocytes have on many occasions been used to define TAA that have then been successfully cloned. Obviously, these are by themselves ineffective at causing tumor regression. Thus, an important part of the present invention is to boost as well as harness these existing immunological resources to convert these into increased immunity to the targeted cancer by exhibiting one or more effective antitumor response(s).

Another therapeutic effect of the present invention would be to prevent metastases of the cancer following the initial cancer treatment. This effect would be particularly beneficial when the cancer treatment is surgery, as it is known that surgery can cause cancer cells, if not completely excised, to migrate to other tissue and organs and proliferate. However, even with other cancer treatments, such as chemotherapy or radiation therapy, embodiments of the present invention would create immunity, or resistance, resulting in potent anti-tumor responses to the targeted cancer and thereby prevent metastases of the cancer that is being treated.

Moreover, embodiments of the present invention can create immunity, or resistance, to a targeted cancer that is distal from the treatment site. Thus, in the case of localized surgery, radiation therapy or even chemotherapy, the immunity that is created from the localized cancer treatment in conjunction with IL-12 administration can result in anti-tumor and immunity effects distal to the site of treatment. In this manner, embodiments of the invention can treat existing metastasis, as well as prevent such occurrences.

Other therapeutic effects of embodiments of the present invention also include the generation of anti-tumor activity within the immunological system. Thus, embodiments of the invention are immunostimulatory, activating the immune system to create a significant anti-tumor effect. Moreover, in the invention it appears that the anti-tumor activity is related to the generation of immunity and resistance to the targeted cancer. See Figure I for a hypothetical model of the anti-tumor and immunological effects of the endogenous vaccine of the present invention.

Still other therapeutic effects of embodiments of the present invention include alleviation of the hematological toxicities associated with the cancer treatment, especially for radiation therapy and chemotherapy. This hematological effect stems from the hematological effects of IL-12 on bone marrow cells, and other organs related to the lymphatic system.

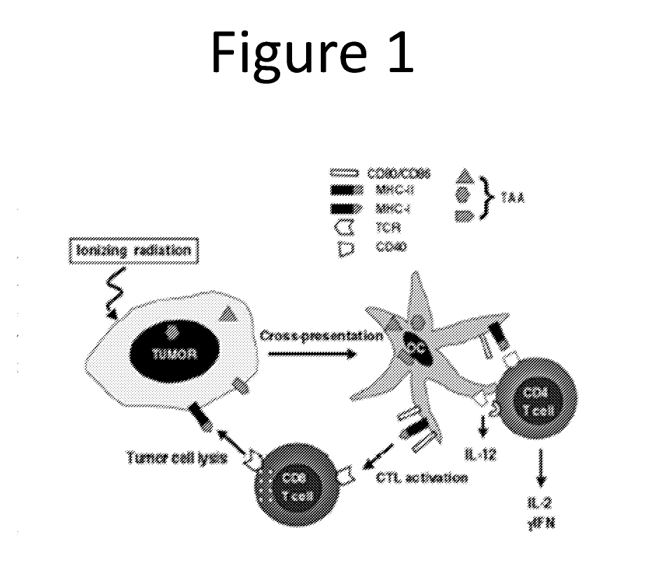

In embodiments of the present invention a component of the endogenous vaccine is the cancer treatment. In these embodiments, the cancer treatment can be any single treatment that kills or destroys cancer cells, including radiation therapy, chemotherapy, and surgery, or combinations of two or more cancer treatments. Generally, the cancer treatment will produce necrosis and/or apoptosis of the cancer cells or tissue being treated. Such necrosis or apoptosis generated by the destruction or death of the cancer cells or tissue by a particular cancer therapy or combinations of cancer therapies generates tumor-associated antigens (TAAs), as described above, and illustrated in Table 2 and FIG. 1.

For embodiments of the present invention that involve radiation as the cancer treatment, the radiation can be localized at or near the site of the tumor, or the radiation can be administered as total body irradiation (TBI). Or in the case of hematopoietic cancers, such as leukemias and lymphomas, the radiation therapy can target the lymphatic system, including the bone marrow. In the embodiments of the invention that involve radiation, generally a therapeutically effective dose of IL-12 will be given shortly before and/or shortly after the radiation therapy. The preferred time intervals for this embodiment of the invention would be either about 24 hours before the radiation and/or about 1 to about 6 hours after the radiation, or at other time points as described herein.

For embodiments of the present invention that involve chemotherapy, the chemotherapy is generally systemic, administered intravenously or by another systemic method. However, chemotherapy can also be localized in the case of solid tumors. The preferred time intervals for this embodiment of the invention would be about 24 hours before chemotherapy and/or about 1 to about 4 days after chemotherapy, or at other time points as described herein.

For embodiments of the present invention that involve surgery, surgery is generally preformed to excise the tumor mass, which is generally a solid tumor, and is performed frequently in lung cancer, colon cancer, breast cancer or the like. Thus, surgery is performed to locally excise a tumor mass and some of the surrounding tissue to ensure complete tumor eradication. In the present invention, generally a therapeutically effective dose of IL-12 would be administered either before or following the surgery. The preferred time intervals for this embodiment of the invention would be either about 24 hours before the surgery and/or about shortly after the surgery, preferably about 6 to about 48 hours after the surgery, or at other time points as described herein. A preferred embodiment of the present invention would involve both the use of surgery and radiation or chemotherapy as the cancer treatment. In this embodiment, a therapeutically effective dose of IL-12 could be administered before and/or after the surgery and/or before and/or after the radiation therapy or chemotherapy.

Another property of the endogenous vaccine or vaccine effect of the present invention is an anti-tumor or anti-cancer effect. This anti-tumor or anti-cancer effect usually will accompany the endogenous vaccine effect, but does not necessarily have to be present. Thus, the present invention can produce an endogenous vaccine effect, which gives rise to increased immunity or resistance to a targeted cancer, but additionally may also give rise to a decrease in or a full remission from the targeted cancer. The anti-tumor or anti-cancer effect may be found in both the treatment of solid tumors, such as breast, lung, colon cancer or the like, and hematopoietic tumors or cancers, such as leukemia, lymphoma or myeloma or the like.

The endogenous vaccine of the present invention may be achieved within the body of a subject by one or more various means. For example, the vaccine of the invention may result in: (a) endogenous antigen producing cells (APC) mobilized to a tumor site or cancer site following administration of IL-12; (b) endogenous antigen producing cells (APC) mobilized to a tumor site or cancer site following administration of IL-12 and the APC are dendritic cells; and/or (c) cytotoxic t-lymphocytes (CTL) produced as a result of the method can comprise one or more cell types selected from the group consisting of CD4⁺ cells and CD8⁺ cells.

The endogenous vaccine effect can comprise: (a) an anti-tumor response; (b) the generation of immunity to the treated cancer; (c) prevention of metastasis of the cancer; (d) treatment of metastasis of the cancer; (e) activation of endogenous antigen producing cells (APC); (f) activation of endogenous antigen producing cells (APC), wherein the APC fuse with the cancerous cells of the subject; (g) activation of endogenous antigen producing cells (APC), wherein the APC are mobilized to a tumor site or cancer site by the one or more cancer treatments or anti-pathogen treatments; (h) activation of endogenous antigen producing cells (APC), wherein the antigen producing cells are mobilized to a tumor site or cancer site by the administration of IL-12; (i) activation of endogenous antigen producing cells (APC) and the antigen producing cells are specific for the cancer; (j) activation of endogenous antigen producing cells (APC) and specific APC are produced from incorporation of one or more cancer-associated antigens into the antigen producing cells, which are then presented as antigens on the antigen producing cells; (k) activation of endogenous antigen producing cells (APC) and the antigen presenting cells are dendritic cells; or (1) any combination thereof. Moreover, the cancer-associated antigen producing cells can promote the production of cytotoxic T cells (CTL), which can comprise CD4⁺ T cells, CD8⁺ T cells, or a combination thereof. In addition, the dendritic cells can be (a) activated by one or more of the cancer treatments; (b) mobilized to tumor sites or cancerous sites with the subject by one or more of the treatments to a tumor site or cancer site, (e) mobilized to a tumor site or cancer site by the administered of IL-12; (f) activated by the administered IL-12; (g) activated by the administered IL-12 and activation of the dendritic cells can involve dendritic cell maturation. Additionally, dendropoiesis can occur at or near a tumor site or cancer site, which can result in the proliferation of dendritic cells at or near a tumor site or cancer site.

Thus, the endogenous vaccine effect can be generated from the activation of endogenous antigen producing cells (APCs). In this scenario, the production of the cancer associated APCs is produced from the incorporation of one or more cancer-associated antigens into the antigen producing cells, which are then presented as antigens on the antigen producing cells. These APCs also may fuse with the cancer cells of the subject during an uptake of cancer associated antigens from the cancer cells. Additionally these APCs may be localized at or near the tumor site. But also, the cancer associated APCs may mobilized to the tumor site by the one or more cancer treatments or IL-12 or any other added cytokine, such as Ft13 ligand, G-CSF or GM-CSF. Further, the cancer associated APC may be generated by the endogenous vaccine of the present invention to be specific for the targeted cancer or may be more generalized and in this later case may provide immunity or resistance to more than one cancer. Additionally, the cancer associated APCs can promote the production of cytotoxic T cells (CTL). These CTL may comprise CD4⁺ T cells and/or CD8⁺ T cells.

In this scenario, the APCs may comprise dendritic cells, which may fuse with the cancer cells of the subject. These dendritic cells may be activated by one or more of the cancer treatments and/or IL-12, and the activation of these dendritic cells results in their maturation and subsequent proliferation. These dendritic cells also can be mobilized by one or more of the cancer treatments and/or IL-12, or another cytokine, such as Ft13 ligand, G-CSF or GM-CSF. Also hematopoietic stem or precursor cells may be mobilized to the tumor site by the components of the endogenous vaccine of the present invention, and this mobilization of hematopoietic stem cell or precursor cells may give rise to hematopoiesis outside of the bone marrow, which in turn may give rise to the proliferation of hematopoietic cells, such as monocytes, macrophages, platelets, lymphocytes, T cells, dendritic cells and neutrophils or the like. These hematopoietic stem cells or precursor cells may also comprise dendritic stem cell or dendritic precursor cells. The mobilization of dendritic stem cell or dendritic precursor cells may also involve dendropoiesis at or near the tumor site, which gives rise to the proliferation of immature dendritic cells at or near the tumor site. In the case of hematopoietic tumors or cancers, the tumor site may be anywhere in the blood, bone marrow, spleen or other hematopoietic organs.

IL-12 Boosters:

In the present invention, an endogenous vaccine is created within the body of the subject. This endogenous vaccine can be boostered at time points subsequent to the initial production of the endogenous vaccine within the subject. In preparation for the generation of a booster, cancer cells can be taken from the subject prior to treatment with a cancer treatment, and are preserved in some manner, such as cyropreservation. To generate the booster, the cancer cells are subject to irradiation and a dose that will cause apoptosis and/or necrosis of the cancer cells. These irradiated cancer cells are then administered to the subject along with IL-12, where the irradiated cells and IL-12 are given at time points that are close in time, such as simultaneously or near simultaneously. This booster will generate the immunity related cells and molecules that will increase resistance to the targeted cancer.

Use of Autologous or Allogenic Cancer Cells:

Still another embodiment of the present invention further comprises using either autologous cells, i.e., from a cancer patient, or allogenic cells, i.e, not from the cancer patient, such as the use of cancer cell line related to the targeted cancer, as a source of tumor associated antigens for the generation of an endogenous vaccine.

In this embodiment, a sufficient number of tumor or cancer cells are used. The number of tumor or cancer cells preferably comprises 1 million cells or more, however, 10,000 cells or less should be sufficient. These autologous or allogenic tumor or cancer cells are then exposed to radiation in a sufficient dose to cause cellular apoptosis and/or necrosis. Preferably the cancer cells are treated with a radiation dose sufficient to cause cellular lysis. The cancer cells also can be irradiated in the presence of other agents that cause the cells to be radiosensitive, thus ensuring complete destruction of the cells.

After generating tumor associated antigens from the cancer cells via radiation, these cells are injected into the patient, preferably by a subcutaneous route. However, other injection routes are applicable. Administration of a therapeutically amount of IL-12 is administered before or after, or before and after, the administration of the irradiated cells to the patient. Administration of IL-12 can be in a single dose or repeated doses. For human subjects a therapeutically effective dose of IL-12 is generally less than 1000 ng/kg/day. Other useful dosages of IL-12 are described herein. In this embodiment, the tumor or cancer cells derived from the patient can be from a solid tumor or hematopoietic tumor. Moreover, this embodiment of the endogenous vaccine of the present invention also comprises administering a therapeutically effective dose of radiation or chemotherapy one or more in times either before or after, or before and after, the injection of the irradiated tumor-containing or cancer-containing cells.

Thus the invention comprising an endogenous vaccine which further comprises the administration of cells, wherein the cells are autologous or allogenic. If the cells are autologous, then the autologous cells can be taken from the spleen of the subject either before or after administration of the endogenous vaccine. Additionally, the autologous cells can be cultured ex vivo and then administered to the subject, where optionally the ex-vivo culture comprises cytokines, and further optionally where the cytokines comprise IL-12. The cells can also be irradiated ex vivo prior to administration. Therefore, the present invention encompasses the administration of a booster given to the subject following the administration of the endogenous vaccine, wherein the booster comprises the administration of cells, the administration of IL-12, or a combination thereof.

Another embodiment of the endogenous vaccine of the present invention includes taking blood cells from the patient prior to treatment. Preferably these blood cells comprise lymphocytes isolated from peripheral blood, or harvested from the bone marrow or spleen. The blood cells, preferably lymphocytes, can be cultured to expand the cells at least two fold using current or future techniques for expansion of blood cells, preferably lymphocytes. After culturing the blood cells, preferably lymphocytes, these cells can be administered near the time of administration, i.e., preferably within one week, either before or after, or before and after, of any one dose of IL-12 used to generate the endogenous vaccine of the present invention, or at other time points as described herein. Further, these cells are to be given back to the patient following any radiation or chemotherapy, if applicable. Optionally, the radiation or chemotherapy is administered after the isolation of lymphocytes from the patient and before the administration of the cultured lymphocytes to the patient. Further, when the blood cells are lymphocytes the culture can be enriched in the population of cytotoxic lymphocytes within the population of lymphocytes. The expansion of blood cells, preferably lymphocytes, can also be performed in the presence of cytokines. See Table 3 (III) for the preferred cytokines to generate cytotoxic T lymphocytes.

The data described in the examples below support the effectiveness of the endogenous vaccine of the invention. Specifically, Example 1 describes data showing that lymphoma-bearing mice treated with IL-12 and radiation had an average tumor size that was 100× less than that observed with mice treated with radiation alone or IL-12 along: e.g., tumor sizes of about 100 mm³ for lymphoma-bearing mice treated with IL-12 and radiation, as compared to an average tumor size of about 10,000 mm³ for lymphoma-bearing mice treated with radiation alone or IL-12 alone. Moreover, Example 2 describes data demonstrating that administration of IL-12 in conjunction with surgery (e.g., removal of tumors) and radiation provides a protective immune response. Specifically, Example 2 describes an experiment in which large tumors were surgically from mice, followed by radiation in first group and radiation+IL12 in a second group. Subsequently, the mice were re-challenged (re-inoculated) with lymphoma cells. As shown in FIG. 2A, all of the control mice (3/3) developed large subcutaneous tumors, whereas for the IL-12-treated group shown in FIG. 2(B), only ⅓ of the IL-12-treated mice developed a relatively small tumor, demonstrating at a mimium a 66% increase in a protective immune response, with a likely response even larger as the tumor observed in the IL-12 mouse was very small as compared to the large tumors observed in the non-IL-12 treated group. These data demonstrate that IL-12 can facilitate both hematopoietic recovery and tumor remission in the clinical setting.

### IV. Embodiments Related to an Endogenous Pathogen Vaccine

A second embodiment of the present invention comprises an endogenous vaccine directed to generating immunity to an exogenous pathogen that is within the body of the subject. The endogenous vaccine of the present invention comprises the following components: (1) radiation therapy, chemotherapy, and/or surgery, which is administered to a subject infected with a pathogen; and (2) administration of a therapeutically effective dose of IL-12 to the subject near the time the therapy of (1) is administered to the subject, and preferably within about 96 hours of the treatment of (1). The combination of the administration of radiation, chemotherapy, and/or surgery, and IL-12 endogenously generates immunity-related cells and molecules in the subject. The radiation, chemotherapy, and/or surgery can cause necrosis and/or apoptosis of cells that harbor the pathogen within the subject. In this embodiment, an anti-pathogenic response is elicited by the endogenous vaccine of the present invention.

The endogenous vaccine of the invention can result in lowering the pathogen load in the subject post-treatment with IL-12 as compared to a subject receiving the same anti-pathogen treatment but which does not receive IL-12. Moreover, following administration of the one or more anti-pathogen treatments and IL-12, the subject can be resistant to recurrence of the pathogen infection. Further, administration of IL-12 can reduces the hematological toxicity of the anti-pathogen treatment, which is significant as the anti-pathogen treatment can cause necrosis and/or apoptosis of cells within the subject that harbor the pathogen.

The subject in the present invention is a mammal, and generally, the subject is a human who is infected with a particular pathogen. For human subjects a therapeutically effective dose of IL-12 is generally less than 1000 ng/kg/day, with other exemplary dosages of IL-12 described herein.

The pathogen can be a microorganism, such as a virus, bacteria, prion, fungi, or yeast. Pathogenic viruses and microorganisms as known in the art. Exemplary pathogenic viruses include, but are not limited to, HIV (e.g., HW-1 and HIV-2) and hepatitis viruses, such as Hepatitis A, Hepatitis B, Hepatitis C, Hepatitis D and Hepatitis G.

If chemotherapy is used, then it can be antibiotic, antiviral, or a combination thereof. If radiation is used, then the radiation can be total body radiation, localized at or near the site of pathogen infection, target the lymphatic system, localized to the thymus, and/or localized to the liver.

Thus, embodiments of the present invention include repeated administration, i.e., more than one administration of IL-12, at certain time intervals following the initial administration. Additionally, the two components can be administered more than once to achieve the desired effect of immunity to the targeted pathogen, as well as eradication of the pathogenic containing cells from the subject. Also each component may be administered more than once with or without the other component to achieve the desired therapeutic effects. Thus, embodiments of the present invention include repeated administration, i.e., more than one administration of IL-12, at certain time intervals following the initial administration.

In the invention, a therapeutically effective dose of IL-12 may be administered one or more times following the initial administration. Generally the components of the invention, namely radiation, chemotherapy, and/or surgery, and IL-12, are administered within about 96 to about 48 hours of each other, or at other time points as described herein. However, the components of the endogenous vaccine of the invention may be administered within about 6 hours of each other or within about 3 hours of each other or even near simultaneously in the case of radiation.

In one exemplary embodiment, a therapeutically effective dose of IL-12 is administered about 24 hours before the pathogenic treatment, namely radiation or chemotherapy. Another exemplary embodiment is where the pathogenic treatment is administered first, followed by a therapeutically effective dose of IL-12 that is administered shortly after the administration of the pathogenic treatment, preferably within about 24 hours to about 48 hours after the pathogenic treatment. In still another preferred embodiment, IL-12 is administered both before and after the pathogenic treatment of radiation or chemotherapy. The therapeutically effective initial dose can be followed with one or more subsequent administrations of IL-12. These subsequent doses may be the same or different from the initial dose.

IL-12 can be administered to the subject in various ways. These methods of administration include intravenous, subcutaneous, intraperitoneal, intranodal, intradermal or the like. Another method of administration of IL-12 is via continuous infusion which would generally be intravenous infusion. The continuous infusion method has the advantage of delivery a low dose of IL-12 over longer time period, which can add to the effectiveness of present invention.

The vaccine of the present invention comprises administering a therapeutically effective dose of IL-12 along with a therapy that can destroy pathogen-containing cells to generate immunity to the internal pathogen, thus eliminating or reducing the pathogen within the body. Also, either component of the endogenous vaccine can be repeated one or more times. In addition, the two components can be repeated one or more times. In subsequent administration of either component of the invention, the dose and time of administration as it relates to the administration of each component can be varied.

The endogenous vaccine produces an anti-pathogenic response, and may generate immunity to the targeted pathogen. This anti-pathogenic response is produced from the activation of endogenous antigen producing cells (APCs), which may fuse with the subject's cells that are harboring the pathogen. The antigen producing cells that are generated by embodiments of the invention may be specific for the particular infectious disease or may be general for several types of infectious diseases. Also, the antigen producing cells can be mobilized to the site of infection by the radiation or chemotherapy or the administration of IL-12.

Thus, the endogenous vaccine of the present invention involves the generation of the pathogen-associated antigen producing cells, where these APCs arise from the incorporation of one or more pathogen-associated antigens into the antigen producing cells, which are then presented as antigens on the antigen producing cells. These pathogen-associated antigen producing cells can then promote the production of cytotoxic T cells, which comprise CD4⁺ T cells and/or CD8⁺ T cells.

Moreover, the APCs that are generated via the endogenous vaccine of the invention in the presence of one or more pathogens may comprise dendritic cells. In this embodiment, the dendritic cells fuse with the cells of the subject that harbor the pathogen. The dendritic cells are activated by the radiation, chemotherapy, and/or surgery, and/or the administration of IL-12. The activation of the dendritic cells may involve dendritic cell maturation.

Further, the dendritic cells may be mobilized to pathogenic sites within the subject by the radiation, chemotherapy, and/or surgery, and/or IL-12 administration. In addition, the dendritic cells are activated by the administered IL-12, and activation of the dendritic cells can involve dendritic cell maturation. Moreover, dendropoiesis can occur at or near a site of infection, and the dendropoiesis can result in the proliferation of dendritic cells at or near a site of infection. Still further, radiation, chemotherapy, and/or surgery can lead to mobilization of dendritic cells or stem cells to the pathogenic sites within the subject. These stem cells may comprise hematopoietic stem cells or hematopoietic precursor cells, or dendritic stem cells or dendritic precursors cells. Moreover, the mobilization of such stem cells to the sites harboring pathogens within the subject may lead to hematopoiesis or dendropoiesis occurring at or near the pathogenic site. In turn, the hematopoiesis or dendropoiesis may result in proliferation of hematopoietic cells, comprising monocytes, macrophages, dendritic cells, platelets, T cells, at or near the pathogenic site. Hematopoiesis or dendropoiesis may lead to activation of dendritic cells via administration of the components of the endogenous vaccine of the invention.

Another property of the endogenous vaccine of the present invention is that the administration of IL-12 also reduces the hematological toxicity of the radiation or chemotherapy utilized.

Still another property of the endogenous vaccine of the invention is that the administration of the vaccine may produce a remission from one or more pathogenic infections. Thus, the endogenous vaccine of the present invention generates resistance to a targeted pathogen, or even an unidentified pathogen, by administering radiation, chemotherapy, and/or surgery to a subject who has an infectious disease; and also administering IL-12 to the subject.

A particular embodiment of the invention includes administering the endogenous vaccine to a subject who is infected with the Human Immunodeficiency Virus (HIV) pathogen (HIV-1 and/or HIV-2). In this particular embodiment, the subject may be administered chemotherapy, radiation, and/or surgery at or near the time of the administration of IL-12, as in the first embodiment of the invention. However, if the pathogenic disease is HIV or Acquired Immunodeficiency Syndrome (AIDS), the chemotherapy directed to the HIV pathogen may be Highly Active Anti-retroviral Therapy (HAART).

In embodiments where the pathogen is HIV, radiation therapy can also be used, as well as other forms of chemotherapy. In the case of radiation therapy, for HW infection the radiation may be directed to organs that harbor a high concentration of the pathogen, such as the thymus, or any other organ in the immune system, such as lymph nodes, spleen, etc.

In another particular embodiment, the pathogen may be the hepatitis virus, including but not limited to Hepatitis A, Hepatitis B, Hepatitis C, Hepatitis D and/or Hepatitis G, or the like. In this particular embodiment, radiation therapy can be localized to the liver of the subject. However, chemotherapy may also be used in the endogenous vaccine of the present invention that is directed to creating immunity to a hepatitis virus.

Patient-Derived Pathogenic Cells:

Still another embodiment of the present invention includes an endogenous vaccine to a pathogen comprising using infected pathogenic cells from a patient as the antigen. This endogenous vaccine comprises taking cells infected with a pathogen from the patient and irradiating these cells. In this embodiment, a sufficient number of pathogenic cells are taken from the patient. The number of cells preferably would comprise 1 million cells or more, however, 10,000 cells or less should be sufficient. These patient-derived pathogenic cells are then exposed to radiation sufficient to cause cellular apoptosis and/or necrosis. Preferably the pathogenic cells are treated with a radiation dose sufficient to cause cellular lysis. The patient-derived pathogenic cells can be irradiated in the presence of other agents that cause the cells to be radiosensitive.

Following the irradiation of the patient-derived pathogenic cells, these cells are injected into the patient, preferably by a subcutaneous route. However, other injection routes are applicable. Administration of a therapeutically amount of IL-12 can be performed before, or after, or before and after, the administration of the irradiated cells to the patient. Administration of IL-12 can be in a single dose or repeated doses. For human subjects a therapeutically effective dose of IL-12 is generally less than 1000 ng/kg and preferably less than 500 ng/kg. However, even lower doses of IL-12 are effective, such as doses of less than 100 ng/kg, especially when more than one dose is administered to the subject at varying time intervals. Other exemplary IL-12 dosages are described herein.

In this embodiment the pathogen can be a virus, such as HW or a hepatitis virus, bacteria or other infectious organism. Moreover, this embodiment of the endogenous vaccine of the present invention also comprises administering a therapeutically effective dose of radiation or chemotherapy related to killing the pathogenic organism one or more in times either before or after, or before and after, the injection of the irradiated tumor cells.

Patient Blood Cells:

Another embodiment of this endogenous vaccine of the present invention includes taking blood cells from the patient prior to treatment. Preferably these blood cells comprise lymphocytes, and more preferably cytotoxic T lymphocytes (CTL). The blood cells, preferably lymphocytes, can be cultured to expand the cells at least two fold using current or future techniques for expansion of blood cells, preferably lymphocytes. After culturing the blood cells, preferably lymphocytes, these cells are to be administered near the time of administration of any one dose of IL-12. Further, these cells are to be given back to the patient following any radiation or chemotherapy, if applicable. Further, when the blood cells are lymphocytes the culture can be enriched for the population of CTL within the population of lymphocytes. The expansion of blood cells, preferably lymphocytes, can also be performed in the presence of cytokines. Preferred cytokines to be added to the culture are IL-12, IL-4 and IL-2. Optionally radiation and/or chemotherapy is administered after the isolation of lymphocytes from the patient and before the administration of the cultured lymphocytes to the patient. See Table 3 (III) for the preferred cytokines to generate cytotoxic T lymphocytes.

Accordingly, the invention encompasses an endogenous vaccine further comprising the administration of cells, wherein the cells are autologous or allogenic. If the cells are autologous, then they can be taken from the spleen of the subject either before or after administration of the endogenous vaccine, and the autologous cells are cultured ex vivo and then administered to the patient, where optionally the ex-vivo culture comprises cytokines, and further optionally where the cytokines comprise IL-12. In another embodiment, the invention encompasses an endogenous vaccine further comprising the administration of cells, wherein the cells comprise anti-pathogen cells taken from the subject prior to administration of the endogenous vaccine, and optionally wherein the anti-pathogen cells taken from the subject are irradiated ex vivo prior to administration. The invention can further comprise administering a therapeutically effective dose of radiation and/or chemotherapy one or more in times either before or after, or before and after, the injection of the irradiated pathogen-containing cells.

The invention encompasses an endogenous vaccine which further comprises the administration of a booster to a subject, wherein the booster regenerates immunity-related cells and molecules endogenously, and the booster comprises pathogen cells taken from the subject prior to the anti-pathogen treatment and which are irradiated prior to administration.

TABLE III
Generation of CTL in the presence of IL-12 and other cytokines^{a}

| Culture Conditions^{b} | P815 + PHA^{c} | P815 noPHA^{d} |
|---|---|---|
| Medium | 0 | 0 |
| IL-2 | 1.3 ± 1 | 0 ± 3 |
| IL-4 | 24 ± 3 | 3 ± 4 |
| IL-9 | −0.3 ± 1 | 0 ± 1 |
| IL-10 | 0 ± 2 | −1 ± 1 |
| IL-13 | 3 ± 1 | −1 ± 3 |
| IL-15 | 1 ± 2 | −0.7 ± 1 |
| IL-2 + IL-4 | 29 ± 4 | 4 ± 1 |
| IL-2 + IL-13 | −1 ± 2 | −2 ± 1 |
| IL-2 + IL-15 | 4 ± 2 | 0 ± 1 |
| IL-2 + IL-12 | 23 ± 7 | 2.6 ± 1 |
| IL-4 + IL-12 | 54 ± 5 | 10.6 ± 2 |
| IL-9 + IL-12 | −1 ± 1 | 0 ± 0 |
| IL-10 + IL-12 | 2 ± 1 | 0 ± 1 |
| IL-13 + IL-12 | 0.7 ± 1 | −0.7 ± 1 |
| IL-15 + IL-12 | 28 ± 1 | 5 ± 1 |
| IL-2 + IL-10 + IL-12 | 25 ± 2 | 2.3 ± 1 |

^{a}IL-2 and costimulatory cytokines support CTL generation; requirement of
lectin for target cell lysis. A variety of cytokines were tested in the absence or
presence of IL-12 for ability to enhance the generation of lectin-stimulated CTL
by thymocyte responders. Culture conditions are same as in Table 1.
^{b}Cytokines were added at the initiation of culture at the following concentrations:
IL-2 at 20 U/ml; IL-4 at 100 U/ml; IL-7 at 100 U/ml; IL-9 at 100 U/ml;
IL-10 at 100 U/ml; IL-12 at 10 U/ml; IL-13 at 100 U/ml; IL-15 at 100 U/ml. All
samples were tested in triplicate, and replicate plates were set up to examine
lysis.
^{c} [symbol] CrP815 targets were added to replicate plates in the presence of 10 μg/ml
of PHA (P815 + PHA) or in the absence of PHA (P815 noPHA). Data are
expressed as the percentage specific release ±SEM for the effectors at the end of
the 72 h culture. See Materials and Methods for other details. Total release =
2400 and spontaneous release = 210. [symbol]
indicates data missing or illegible when filed

### V. Model of Possible Vaccine Action

FIG. 1 shows a model for the role for a therapy, such as ionizing radiation, chemotherapy or surgery, in promoting cross-presentation of tumor associated antigens (TAA) and activation of T cells. It is well established that dendritic cells (DC) can efficiently uptake TAA from apoptotic and necrotic tumor or diseased cells and present them to both CD4⁺ and CD8⁺ cytolytic T cells (CTL), a process termed cross-presentation. By killing tumor or diseased cells, the treatment can promote this process. In the presence of adequate “danger signals” that induce DC maturation and up-regulation of co-stimulatory molecules CD80 and CD86, namely the administration of IL-12, tumor or disease-specific T cells are activated to produce proinflammatory cytokines and become effectors capable of killing the tumor or diseased cells. Recognition and killing of tumor or diseased cells by CTL might be further enhanced by the radiation-induced up-regulation of Fas and/or major histocompatibility complex class I (MHC I) molecules on the tumor cells. TCR=T cell receptor; IL=interleukin; IFN=interferon.

### EXAMPLES

### Example 1

### Tumor Size and Hematopoietic Recovery in Lymphomic Mice Receiving Radiation and IL-12

An experiment was conducted to assess the hematopoietic recovery and anti-tumor properties of IL-12 in lymphoma-bearing mice. High sublethal radiation conditions were chosen for this experiment (6.3 Gy), rather than lethal conditions, to ensure that the mice could be observed for at least 30 days post-radiation.

Mice were first inoculated subcutaneously at the back with 1×10⁵ cells using the EL4 cell lymphoma line (ATCC# TIB-39). After one day, mice were then treated with PBS or IL-12 (100 ng in PBS) via tail vein (intravenous) injection. After 24 hours, all mice were irradiated. Mice were subsequently treated with once weekly doses of PBS or IL-12 (30 ng in PBS) for three weeks (3 subsequent doses after radiation).

Hematopoietic recovery and tumor size were assessed in C57BL/6J mice. IL-12 treatment produced significant neutrophil and platelet recovery in lymphoma-bearing mice. Neutrophil recovery for mice treated with IL-12 and radiation reached normal levels by about day 15 during the 30 observation post-radiation period, whereas for control mice (PBS/radiation), extreme neutrophilia (˜50 times normal levels) was observed by day 30. The observed neutrophilia is indicative of the high tumor burden for control mice (46-51). Platelet counts for both the IL-12 group and the control reached normal levels by about day 30 post-radiation. However, for the IL-12 group, the platelet count nadir was attenuated by 43%. No significant differences for red blood cell counts were observed for IL-12-treated and controls, as the red blood cells counts for both groups remained close to normal throughout the post-radiation period.

Tumor size was also evaluated. The lymphoma-bearing mice treated with IL-12 and radiation had an average tumor size of about 100 mm³, whereas lymphoma-bearing mice treated with radiation alone or IL-12 alone had an average tumor size of about 10,000 mm³ (mm³=longest length×shortest length) at 30 days post-radiation.

The experiment showed that IL-12 and radiation significantly affected tumor growth in lymphoma-bearing mice. Specifically, the study showed that dramatic anti-tumor effects are observed for the combinatorial treatment of IL-12 and radiation.

### Example 2

### Tumor Regrowth in Mice Treated with IL-12 and Radiation and Re-Challenged with Lymphoma Cells

In a separate study, the immunological aspects of IL-12 and radiation treatment were assessed. A murine lymphoma tumor model was developed as described above. After developing large tumors (5000 mm³), these lymphoma-bearing mice were given curative treatments by surgically removing the subcutaneous tumors. After recovery from surgery (5 days), mice were then treated with either radiation (control group) or radiation and 100 ng of IL-12 (IL-12 group). The radiation dose was 6.3 Gy. Mice were then observed for 3 months. No tumor growth was observed in both groups.

After 3 months, all mice were re-challenged (re-inoculated) with lymphoma cells. As shown in FIG. 2A, all control mice (3/3) developed large subcutaneous tumors, whereas for IL-12-treated group shown in FIG. 2(B), only ⅓ of the IL-12-treated mice developed a relatively small tumor.

These results suggest that immunity is generated in lymphoma-bearing mice treated with IL-12 and radiation. The observed immunity effect may be important to the treatment of minimal residual disease (MRD) following myeloablative therapy for hematological malignancies. Overall these experiments show that IL-12 can facilitate both hematopoietic recovery and tumor remission in the clinical setting.

### Example 3

### Comparison of Formulations of IL-12 in Irradiated Mice

An experiment to further investigate the radiomitigation properties of murine IL-12 (rmIL-12) was conducted using two formulations, a sucrose and trehalose-based formulation. Three doses of murine rmIL-12 were tested using either formulation, along with the respective vehicle control group. The doses investigated were 2, 18 and 162 ng and compared to vehicle alone.

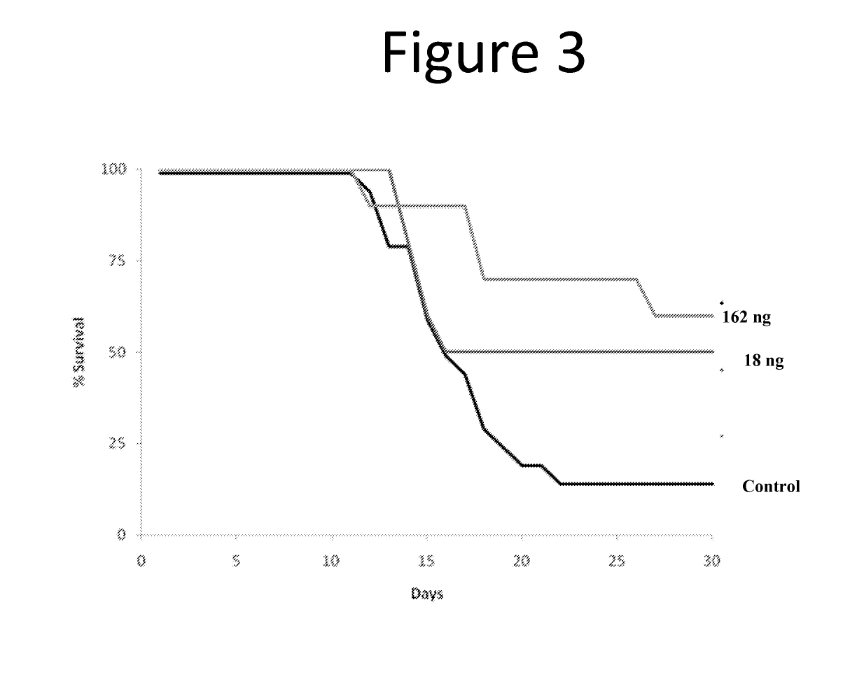

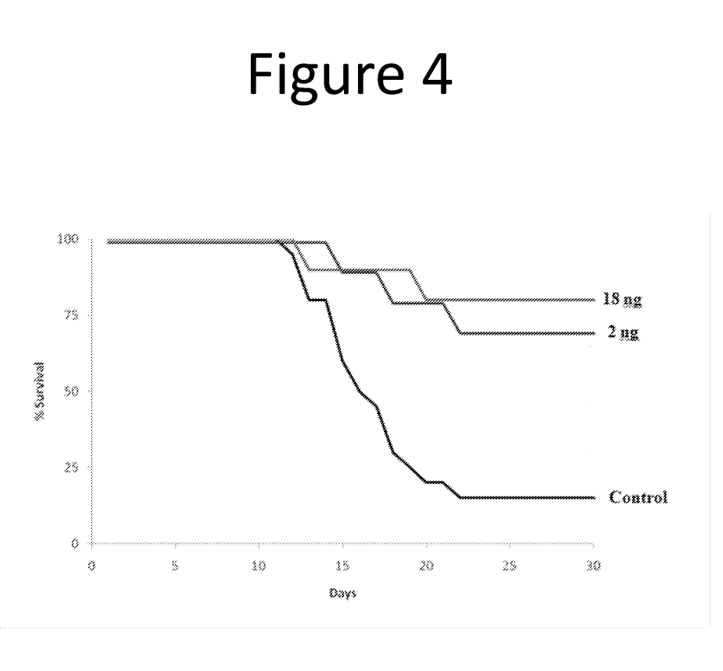

Mice were injected SC with rmIL-12 at 24 hours after exposure to 7.9 Gy. Kaplan-Meier (K-M) plots are shown in FIG. 3 for rmIL-12 in the sucrose-based formulation and in FIG. 4 for RmIL-12 in the trehalose-based formulation.

As depicted in FIGS. 3 and 4, rmIL-12 in either formulation produced potent radiomitigation effects. The overall survival (defined as % group survival) for rmIL-12 in the sucrose formulation (FIG. 1) was 50% at 18 ng and 60% at 162 ng (p<0.05, Fisher exact probability test) (LD85₃₀). For trehalose-formulated rmIL-12 (FIG. 2), the overall survival was 70% at 2 ng (p<0.02, Fisher test) and 80% at 18 ng (p<0.005, Fisher test) (LD85₃₀).

Although the 2 ng dose of rmIL-12 in the sucrose-based formulation produced a modest increase in percent group survival, this dose was not significantly different from control survival either by Kaplan-Meier analysis of survival time or chi square analysis of % group survival. Similarly, although a modest increase in group survival was observed for the 162 ng dose in the trehalose-based formulation, survival was not significantly different from control survival percentage or survival time.

In contrast, the 162 ng dose of rmIL-12 in the sucrose formulation significantly elevated both % group survival (Fisher test, p<0.05) and survival time (K-M analysis, p<0.001) over the control. The 18 ng rmIL-12 formulation in sucrose elevated survival time over controls (K-M, p<0.04), but barely missed elevating % group survival (Fisher test, n.s). RmIL-12 at 2 ng in the trehalose formulation elevated both % survival (Fisher test, p<0.02) and marginally elevated survival time (K-M analysis, p<0.07). The 18 ng dose of IL-12 elevated both % group survival (Fisher test, p<0.005) and survival time (K-M analysis, p<0.03).

A two factor analysis of variance (ANOVA) was performed on the survival times from the K-M analyses. The factors were dose and formulation type. Both factors were highly significant (p<0.01), but the Dose X Formulation interaction was not. The significant formulation factor indicated the surprising and unexpected result that survival time was longer in the trehalose formulation as compared to the sucrose formulation when administered at the same doses. These results suggest greater potency for the trehalose formulation, which is in agreement with the K-M and chi square analyses. The significant dose factor suggests that survival time is dose-related, although maximal survival time is higher with the trehalose formulation (FIG. 3 (survival time vs. dose) and FIG. 4 (survival time vs. formulation)).

Although rmIL-12 provided statistically significant radiomitigation effects using either the sucrose or the trehalose formulation, rmIL-12 formulation in trehalose added an additional benefit in that this formulation lowers the effective dose required for radiomitigation effects of rmIL-12 about 9-10-fold, i.e., rmIL-12 stabilized by trehalose increases its potency.

rmIL-12 formulated in trehalose allows a targeted, low human dose, which is 100 ng/kg (the 2 ng murine dose can be converted to approximately an 8 ng/kg human dose and the 18 ng murine dose converts to about 72 ng/kg human dose). Further, the data support the notion that the use of trehalose as the formulation for human IL-12 will likely increase the safety profile of the drug during clinical trials.

In conclusion, rmIL-12 possesses potent radiomitigation effects when administered 24 hours after lethal irradiation using two different formulations, namely a sucrose/mannitol formulation (pH 5.6) and trehalose-based formulation (pH 5.6). Unexpectedly and surprisingly, the trehalose formulation significantly increases potency of rmIL-12 relative to the sucrose/mannitol formulation.

The above examples are given to illustrate the present invention. It should be understood, however, that the spirit and scope of the invention is not to be limited to the specific conditions or details described in these examples. All publicly available documents referenced herein, including but not limited to U.S. patents, are specifically incorporated by reference.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A method of treating a subject having cancer, the method comprising:
(a) administering one or more treatments to a subject with cancer, wherein the cancer treatments are selected from the group consisting of radiation therapy, chemotherapy, surgery, and a combination thereof; and
(b) administering a therapeutically effective dose of IL-12 to the subject within about 96 hours of the one or more cancer treatments;
wherein administration of the one or more cancer treatments and IL-12 endogenously generates immunity-related cells and molecules in the subject.

2. A method of treating a subject infected with a pathogen, the method comprising:
(a) administering one or more anti-pathogen treatments to a subject infected with a pathogen, wherein the anti-pathogen treatments are selected from the group consisting of radiation therapy, chemotherapy, surgery, or any combination thereof; and
(b) administering a therapeutically effective dose of IL-12 to a subject within about 96 hours of the one or more anti-pathogen treatments;
wherein administration of the one or more anti-pathogen treatments and IL-12 endogenously generates immunity-related cells and molecules in the subject.

3. The method of claim 1, wherein the cancer is a solid tumor, a hematopoietic cancer, or a combination thereof.

4. The method of claim 1, wherein:
(a) the method prolongs cancer remission in subjects with cancer;
(b) the subject is resistant to recurrence of the cancer;
(b) the incidence of new tumors in the subject at about 3 months post-treatment with IL-12 is lower than that of a subject receiving the same cancer treatment but which does not receive IL-12;
(c) the average volume of tumors in the subject at about 3 months post-treatment with IL-12 is less than that of a subject receiving the same cancer treatment but which does not receive IL-12; or
(d) any combination thereof.

5. The method of claim 1, wherein:
(a) endogenous antigen producing cells (APC) are mobilized to a tumor site, cancer site, or site of infection following administration of IL-12;
(b) endogenous antigen producing cells (APC) are mobilized to a tumor site, cancer site, or site of infection following administration of IL-12 and the APC are dendritic cells; and/or
(c) cytotoxic t-lymphocytes (CTL) produced as a result of the method comprise one or more cell types selected from the group consisting of CD4⁺ cells and CD8⁺ cells.

6. The method of claim 2, wherein the pathogen is:
(a) a microorganism;
(b) a microorganism, and the microorganism is a virus, bacteria, prion, fungi, or yeast;
(c) a virus and the virus is HIV;
(d) a virus and the virus is a hepatitis virus; or
(e) a virus and the virus is a hepatitis virus, and the hepatitis virus is selected from the group consisting of Hepatitis A, Hepatitis B, Hepatitis C, Hepatitis D and Hepatitis G.

7. The method of claim 2, wherein:
(a) the pathogen load in the subject post-treatment with IL-12 is lower than that of a subject receiving the same anti-pathogen treatment but which does not receive IL-12;
(b) following administration of the one or more anti-pathogen treatments and IL-12, the subject is resistant to recurrence of the pathogen infection; or
(c) any combination thereof.

8. The method of claim 1, wherein the subject is mammal, such as a human.

9. The method of claim 1, wherein the dose of IL-12 is less than about 1000 ng/kg.

10. The method of claim 1, further comprising:
(a) removing lymphocytes from the spleen of the subject,
(b) culturing the lymphocytes following the one or more treatments,
(c) reintroducing the cultured lymphocytes into the subject, and optionally
(d) where the lymphocytes are cultured in step (b) in the presence of one or more cytokines selected from the group consisting of IL-2, IL-4, and IL-12.

11. The method of claim 1, wherein:
(a) IL-12 is additionally administered one or more times following the initial IL-12 administration;
(b) the cancer treatments or anti-pathogen treatments are repeated one or more times following the initial treatment; or
(c) a combination thereof.

12. The method of claim 1, further comprising the administration of a booster to the subject, wherein the booster regenerates immunity-related cells and molecules endogenously, and the booster comprises cancer or pathogen cells taken from the subject prior to the cancer or anti-pathogen treatment and which are irradiated prior to administration.

13. A vaccine created endogenously within the body of a subject comprising the following components:
(a) one or more treatments which are radiation therapy, chemotherapy, surgery, or a combination thereof, which are administered to a subject having cancer or a subject infected with a pathogen; and
(b) a therapeutically effective dose IL-12, which is administered to the subject within about 96 hours of the one or more treatments;
wherein the combination of (a) and (b) produces an endogenous vaccine to the pathogen or cancer in the subject.

14. An endogenous vaccine comprising:
(a) obtaining tumor or cancer-containing or pathogen-containing cells from a subject;
(b) irradiating the tumor or cancer-containing or pathogen-containing cells;
(c) injecting the irradiated tumor or cancer-containing or pathogen-containing cells into the subject; and
(d) administering one or more therapeutically effective dose(s) of IL-12 to the subject within about 96 hours of the injection of cells,
wherein the vaccine results in the generation of immunity-related cells and molecules in the subject to the pathogen or cancer.

15. The vaccine of claim 13, wherein the subject is a mammal, such as a human.

16. The vaccine of claim 13, wherein the cancer is a solid tumor, a hematopoietic cancer, or a combination thereof.

17. The vaccine of claim 13, wherein the pathogen is:
(a) a microorganism;
(b) a microorganism, and the microorganism is a virus, bacteria, prion, fungi, or yeast;
(c) a virus and the virus is HIV;
(d) a virus and the virus is a hepatitis virus; or
(e) a virus and the virus is a hepatitis virus, and the hepatitis virus is selected from the group consisting of Hepatitis A, Hepatitis B, Hepatitis C, Hepatitis D and Hepatitis G.

18. The vaccine of claim 13, wherein the effective dose of IL-12 is less than about 1000 ng/kg.

19. The vaccine of claim 13, wherein:
(a) IL-12 is administered one or more times following the initial IL-12 administration;
(b) the cancer treatments or anti-pathogen treatments are repeated one or more times; or
(c) any combination thereof.

20. The vaccine of claim 13, wherein:
(a) the radiation therapy is localized at or near the site of a tumor or cancer or a site of pathogen infection;
(b) the radiation therapy is total body radiation;
(c) the radiation therapy targets the lymphatic system;
(d) the radiation therapy is localized to the thymus;
(e) the radiation therapy is localized to the liver;
(f) the chemotherapy is HAART therapy directed to the HIV virus;
(g) the chemotherapy is antibiotic, antiviral, or a combination thereof; or
(h) any combination thereof.

21. The vaccine of claim 13, wherein the endogenous vaccine effect comprises:
(a) an anti-tumor or anti-pathogenic response;
(b) the generation of immunity to the treated cancer or pathogen;
(c) prevention of metastasis of the cancer;
(d) treatment of metastasis of the cancer;
(e) activation of endogenous antigen producing cells (APC);
(f) activation of endogenous antigen producing cells (APC), wherein the APC fuse with the pathogenic or cancerous cells of the subject;
(g) activation of endogenous antigen producing cells (APC), wherein the APC are mobilized to a tumor site, cancer site, or site of infection by the one or more cancer treatments or anti-pathogen treatments;
(h) activation of endogenous antigen producing cells (APC), wherein the antigen producing cells are mobilized to a tumor site, cancer site, or site of infection by the administration of IL-12;
(i) activation of endogenous antigen producing cells (APC) and the antigen producing cells are specific for the cancer or infectious disease;
(j) activation of endogenous antigen producing cells (APC) and specific APC are produced from incorporation of one or more cancer-associated antigens, or pathogen-associated antigens, into the antigen producing cells, which are then presented as antigens on the antigen producing cells;
(k) activation of endogenous antigen producing cells (APC) and the antigen presenting cells are dendritic cells; or
(l) any combination thereof.

22. The vaccine of claim 21, wherein:
(a) the cancer-associated antigen producing cells, or pathogen-associated antigen producing cells, promote the production of cytotoxic T cells (CTL);
(b) the cancer-associated antigen producing cells, or pathogen-associated antigen producing cells, promote the production of cytotoxic T cells (CTL) and the CTL comprise CD4⁺ T cells, CD8⁺ T cells, or a combination thereof;
(c) the dendritic cells are activated by one or more of the cancer treatments or anti-pathogen treatments;
(d) the dendritic cells are mobilized to pathogenic sites, tumor sites, or cancerous sites with the subject by one or more of the cancer or anti-pathogen treatments to a site of infection, tumor site, or cancer site,
(e) the dendritic cells are mobilized to a tumor site, cancer site, or site of infection by the administered of IL-12;
(f) the dendritic cells are activated by the administered IL-12;
(g) the dendritic cells are activated by the administered IL-12 and activation of the dendritic cells involves dendritic cell maturation;
(h) dendropoiesis occurs at or near a tumor site, cancer site, or site of infection;
(i) dendropoiesis occurs at or near a tumor site, cancer site, or site of infection and the dendropoiesis results in the proliferation of dendritic cells at or near a tumor site, cancer site, or site of infection; or
(j) any combination thereof.

23. The vaccine of claim 13, wherein:
(a) administration of IL-12 reduces the hematological toxicity of the cancer treatment or anti-pathogen treatment;
(b) the cancer treatment or anti-pathogen treatment causes necrosis and/or apoptosis of cells within the subject that harbor the pathogen or cancer; or
(c) any combination thereof.

24. The vaccine of claim 13, further comprising:
(a) the addition of interferon alpha.
(b) the administration of cells;
(c) the administration of cells, wherein the cells are autologous or allogenic;
(d) the administration of cells, wherein the cells are autologous and:
(i) the autologous cells are taken from the spleen of the subject either before or after administration of the endogenous vaccine;
(ii) the autologous cells are cultured ex vivo and then administered to the subject, where optionally the ex-vivo culture comprises cytokines, and further optionally where the cytokines comprise IL-12; or
(iii) a combination thereof;
(e) the administration of cells, wherein the cells comprise cancer cells or anti-pathogen cells taken from the subject prior to administration of the endogenous vaccine, and optionally wherein the cancer cells or anti-pathogen cells taken from the subject are irradiated ex vivo prior to administration; or
(f) any combination thereof.

25. The vaccine of claim 13, further comprising the administration of a booster to the subject following the administration of the endogenous vaccine, wherein the booster comprises the administration of cells, the administration of IL-12, or a combination thereof.
